SEQUENCE LISTING (7 sequences)

SEQ ID NO: 1 - protein, 715 aa, HUMAN
MAANMYRVGDYVYFENSSSNPYLIRRIEELNKTANGNVEAKVVCFYRRRDISSTLIALADKHATLSVCYKAGPGADNGEEGEIEEEMENPEMVDLPEKLKHQLRHRELFLSRQLESLPATHIRGKCSVTLLNETESLKSYLEREDFFFYSLVYDPQQKTLLADKGEIRVGNRYQADITDLLKEGEEDGRDQSRLETQVWEAHNPLTDKQIDQFLVVARSVGTFARALDCSSSVRQPSLHMSAAAASRDITLFHAMDTLHKNIYDISKAISALVPQGGPVLCRDEMEEWSASEANLFEEALEKYGKDFTDIQQDFLPWKSLTSIIEYYYMWKTTDRYVQQKRLKAAEAESKLKQVYIPNYNKPNPNQISVNNVKAGVVNGTGAPGQSPGAGRACESCYTTQSYQWYSWGPPNMQCRLCASCWTYWKKYGGLKMPTRLDGERPGPNRSNMSPHGLPARSSGSPKFAMKTRQAFYLHTTKLTRIARRLCREILRPWHAARNPYLPINSAAIKAECTARLPEASQSPLVLKQAVRKPLEAVLRYLETHPRPPKPDPVKSVSSVLSSLTPAKVAPVINNGSPTILGKRSYEQHNGVDGNMKKRLLMPSRGLANHGQTRHMGPSRNLLLNGKSYPTKVRLIRGGSLPPVKRRRMNWIDAPGDVFYMPKEETRKIRKLLSSSETKRAARRPYKPIALRQSQALPPRPPPPAPVNDEPIVIED

SEQ ID NO: 2 - DNA, 2662 bp, HUMAN
cccggacatggccgccaacatgtacagggtcggagactacgtctactttgagaactcctccagcaacccatacctgatccggagaatcgaggagctcaacaagacggccaatgggaacgtggaggccaaagtggtgtgcttctaccggaggcgggacatctccagcaccctcatcgccctggccgacaagcacgcaaccctgtcagtctgctataaggccggaccgggggcggacaacggcgaggaaggggaaatagaagaggaaatggagaatccggaaatggtggacctgcccgagaaactaaagcaccagctgcggcatcgggagctgttcctctcccggcagctggagtctctgcccgccacgcacatcaggggcaagtgcagcgtcaccctgctcaacgagaccgagtcgctcaagtcctacctggagcgggaggatttcttcttctattctctagtctacgacccacagcagaagaccctgctggcagataaaggagagattcgagtaggaaaccggtaccaggcagacatcaccgacttgttaaaagaaggcgaggaggatggccgagaccagtccaggttggagacccaggtgtgggaggcgcacaacccactcacagacaagcagatcgaccagttcctggtggtggcccgctctgtgggcaccttcgcacgggccctggactgcagcagctccgtccgacagcccagcctgcacatgagcgccgcagctgcctcccgagacatcaccctgttccacgccatggatactctccacaagaacatctacgacatctccaaggccatctcggcgctggtgccgcagggcgggcccgtgctctgcagggacgagatggaggagtggtctgcatcagaggccaaccttttcgaggaagccctggaaaaatatgggaaggatttcacggacattcagcaagattttctcccgtggaagtcgctgaccagcatcattgagtactactacatgtggaagaccaccgacagatacgtgcagcagaaacgcttgaaagcagctgaagctgagagcaagttaaagcaagtttatattcccaactataacaagccaaatccgaaccaaatcagcgtcaacaacgtcaaggccggtgtggtgaacggcacgggggcgccgggccagagccctggggctggccgggcctgcgagagctgttacaccacacagtcttaccagtggtattcttggggtccccctaacatgcagtgtcgtctctgcgcatcttgttggacatattggaagaaatatggtggcttgaaaatgccaacccggttagatggagagaggccaggaccaaaccgcagtaacatgagtccccacggcctcccagcccggagcagcgggagccccaagtttgccatgaagaccaggcaggctttctatctgcacacgacgaagctgacgcggatcgcccggcgcctgtgccgtgagatcctgcgcccgtggcacgctgcgcggaacccctacctgcccatcaacagcgcggccatcaaggccgagtgcacggcgcggctgcccgaagcctcccagagcccgctggtgctgaagcaggcggtacgcaagccgctggaagccgtgcttcggtatcttgagacccacccccgcccccccaagcctgaccccgtgaaaagcgtgtccagcgtgctcagcagcctgacgcccgccaaggtggcccccgtcatcaacaacggctcccccaccatcctgggcaagcgcagctacgagcagcacaacggggtggacggcaacatgaagaagcgcctcttgatgcccagtaggggtctggcaaaccacggacagaccaggcacatgggaccaagccggaacctcctgctcaacgggaagtcctaccccaccaaagtgcgcctgatccgggggggctccctgcccccagtcaagcggcggcggatgaactggatcgacgccccgggtgacgtgttctacatgcccaaagaggagaccaggaagatccgcaagctgctctcatcctcggaaaccaagcgtgctgcccgccggccctacaagcccatcgccctgcgccagagccaggccctgccgccgcggccaccgccacctgcgcccgtcaacgacgagcccatcgtcatcgaggactaggggccgcccccacctgcggccgccccccgcccctcgcccgcccacacggccccttcccagccagcccgccgcccgcccctcagtttggtagtgccccacctcccgccctcacctgaagagaaacgcgctccttggcggacactgggggaggagaggaagaagcgcggctaacttattccgagaatgccgaggagttgtcgtttttagctttgtgtttactttttggctggagcggagatgaggggccaccccgtgcccctgtgctgcggggccttttgcccggaggccgggccctaaggttttgttgtgttctgttgaaggtgccattttaaattttatttttattactttttttgtagatgaacttgagctctgtaacttacacctggaatgttaggatcgtgcggccgcggccggccgagctgcctggcggggttggcccttgtcttttcaagtaattttcatattaaacaaaaacaaagaaaaaaaatcttataaaaaggaaaa

SEQ ID NO: 3 - protein, 668 aa, Human
MAANMYRVGDYVYFENSSSNPYLVRRIEELNKTANGNVEAKVVCLFRRRDISSSLNSLADSNAREFEEESKQPGVSEQQRHQLKHRELFLSRQFESLPATHIRGKCSVTLLNETDILSQYLEKEDCFFYSLVFDPVQKTLLADQGEIRVGCKYQAEIPDRLVEGESDNRNQQKMEMKVWDPDNPLTDRQIDQFLVVARAVGTFARALDCSSSIRQPSLHMSAAAASRDITLFHAMDTLQRNGYDLAKAMSTLVPQGGPVLCRDEMEEWSASEAMLFEEALEKYGKDFNDIRQDFLPWKSLASIVQFYYMWKTTDRYIQQKRLKAAEADSKLKQVYIPTYTKPNPNQIISVGSKPGMNGAGFQKGLTCESCHTTQSAQWYAWGPPNMQCRLCASCWIYWKKYGGLKTPTQLEGATRGTTEPHSRGHLSRPEAQSLSPYTTSANRAKLLAKNRQTFLLQTTKLTRLARRMCRDLLQPRRAARRPYAPINANAIKAECSIRLPKAAKTPLKIHPLVRLPLATIVKDLVAQAPLKPKTPRGTKTPINRNQLSQNRGLGGIMVKRAYETMAGAGVPFSANGRPLASGIRSSSQPAAKRQKLNPADAPNPVVFVATKDTRALRKALTHLEMRRAARRPNLPLKVKPTLIAVRPPVPLPAPSHPASTNEPIVLED

SEQ ID NO: 4 - DNA, 3060 bp, HUMAN
tccggaaggaggcgaaccctgaggcgggcccggcaagccttccctgcggccggcagagcccaacgactagtgggactccgcgggggcgggggtagctggagcctggctctggcctggcaggagccgagcttgttccggaagaagccgagcggacgggggccagcctcagcgtcccgggagtgaggcgatagctgcggcggcgacagcgcgggccgggatgaaccgcgacggctgaggcagcggaggtgccggctgcgcgggccccagtgagactccctcgaagcggcagcccaccgttcggggctttgcctcgagccgagccctgcccccgcgagcctcccggacccctttgtgcggccggaggcggcggcgggaacggccatggcggccaacatgtaccgggtgggagattacgtctattttgagaactcttccagcaatccttacctggttagacggattgaggagctcaacaagactgcaaatggaaatgtggaggcaaaggttgtctgtcttttccggcgcagggacatttctagtagcctcaacagcctggctgatagtaatgccagggagtttgaagaggaatcaaagcagccaggggtgtctgagcagcagcgccatcaactgaagcaccgggaactttttctttctcggcaatttgaatcattaccagccacccacatacgggggaaatgcagtgtgaccctcttgaatgagacagatatcttgagccagtacctggaaaaggaggactgctttttttactcactggtgtttgaccccgtgcagaagacacttctcgctgatcagggcgagattagagttggttgcaaataccaagctgagatcccagatcgcctagtagagggagaatctgataatcggaaccagcagaagatggagatgaaggtctgggacccagacaaccctctcacagaccggcagatcgaccagtttcttgtggtggcccgagctgtgggaacctttgcaagagccctagattgtagcagctccattcggcagccaagcttgcacatgagtgcagctgctgcctcccgagatatcactctgtttcacgccatggataccttgcaaaggaacggctacgacctggctaaggccatgtcgaccctggtaccccagggaggcccggtgctgtgtcgggatgagatggaggaatggtcagcctcagaggccatgctatttgaggaggccctagagaagtatgggaaggacttcaatgatattcgccaggattttctaccctggaagtcacttgccagcatagtccagttttattacatgtggaaaaccacagaccggtatattcagcagaaaaggttgaaagctgctgaagcagacagcaaactgaaacaggtctacattcccacctacactaagccaaaccctaaccagatcatttctgtgggttcaaaacctggcatgaatggggctggatttcagaagggcctgacttgtgagagttgccacaccacacagtctgctcagtggtatgcctggggcccacctaacatgcagtgccgcctctgtgcttcctgttggatctactggaagaagtatgggggactgaagaccccaactcagcttgagggggccactcggggcaccacggagccacactcaaggggtcatttatccagacctgaagctcaaagtctctctccttacacaaccagcgccaacagggccaagctactggctaagaacagacaaactttcctgcttcagaccacaaagctgacccgtcttgccagacgcatgtgcagggacctattacagccaaggagggccgcccgacggccttatgctcctatcaatgccaatgccatcaaagcagagtgctccattcgacttcctaaggccgccaagactccattgaagattcaccctctggtgcggctgcccctggcaactatcgtcaaagatctggtggcccaggcacccctgaaaccaaaaacacctcggggtaccaagacaccgatcaacagaaaccagctgtcccagaaccggggactggggggcattatggtgaaacgggcctatgagactatggcaggggcaggggttcctttctctgccaatggaaggcctctggcttcagggattcgttcaagctcacagccagcagccaagcgtcagaaactaaacccagctgatgcccccaatcctgtggtgtttgtggccacaaaggataccagggccctacggaaggctctgacccatctggaaatgcggcgagctgctcgccgacccaacttgcccctgaaggtgaagccaacgctgattgcagtgcggccccctgtccctctacctgcaccctcacatcctgccagcaccaatgagcctattgtcctggaggactgagcacctgtggggaagggaggtgggctgagaggtagagggtggatgcccagggcacccaaacctcccttccctttcgtgtcgaagggagtgaggagtgaattaaggaagagagcaagtgagtgtgtgtccctggaggggttgggcgccctctggtgttaccacctcgagacttgtctcatgcctccatgcttgccgatggaggacagactgcaggaacttggcccatgtgggaacctagcctgttttggggggtaggacccacagatgtcttggacagttttggggggagggttttttaattttttaaaagttttgcctccctttgtgaaaggggatggggaggggaagagtaaacagataacaggtggtggtacctggttgggggaggggggcgtgcactgccatgtctttttttttttttttttttttttttttcctaattgggggtttctctttctgtccggtgtccggactttcctaattggagtttgaggcccctaagctggcatcaaccccaggccacgctcgctctttccttccctcccctccccctctgccttttgtacgccagttctcagaaataaagatcttttgtccgtttttttaacctcggattctgtaattggttcttatagtaacaaataaaaagctgttttcttcagcttctcctgg

SEQ ID NO: 5 - DNA, 718 bp, HUMAN
tacatgtggaaaactactgacagatatgtgcaacagaaacgtctaaaagcagcagaagctgagagtaaactgaaacaagtatatatcccaacctacaaaccaaatcccaaccaaatatccactagtaatgggaagcctggtgctgtgaatggagctgtggggaccacgttccagcctcagaatcctctcttagggagagcctgtgagagctgctatgctacacagtctcaccagtggtattcttggggcccacctaatatgcagtgtagattatgtgcaatttgttggctttattggaaaaaatatggaggcttgaaaatgcccacccagtcagaagaagagaagttatctcctagcccaactacagaggaccctcgtgttagaagtcacgtgtcccgccaggccatgcagggaatgccagtccgaaacactgggagtccaaagtctgcagtgaagacccgccaagctttcttccttcatactacatatttcacaaaatttgctcgtcaggtctgcaaaaataccctccggctgcggcaggcagcaagacggccgtttgttgctattaattatgctgccattagggcagaatgtaagatgcttttaaattcttaaccttatatgttgtgcttctgaccattttctcttttcctctctttccttttttttttgtttgtttgtttgcaataaacataagttcttgtgtacaaaaaaaaaa

SEQ ID NO: 6 - protein, 17 aa, Artificial Sequence
  Synthetic Peptide
ENPEMVDLPEKLKHQLR

SEQ ID NO: 7 - protein, 20 aa, Artificial Sequence
  Synthetic Peptide
IDAPGDVFYMPKEETRKIRK

(19) United States
(12) Patent Application Publication
(10) Pub. No.: US 2005/0176072 A1
(43) Pub. Date: Aug. 11, 2005

(54) MTA1 is a predictive and prognostic factor in human breast cancer

(76) Inventors: Michelle D. Martin, Nashville, TN (US); Peter O'Connell, Richmond, VA (US); D. C. Allred, Houston, TX (US); Gary Clark, Boulder, CO (US)

Correspondence Address: FULBRIGHT & JAWORSKI, LLP, 1301 MCKINNEY, SUITE 5100, HOUSTON, TX, 77010-3095, US

(21) Appl. No.: 10/600,747

(22) Filed: Jun. 20, 2003

Related U.S. Application Data
(60) Provisional application No. 60/390,794, filed on Jun. 21, 2002.

(51) Int. Cl. G01N 33/574; C12Q 1/68

(52) U.S. Cl. 435/7.23; 435/6

(57) ABSTRACT

The present invention is directed to providing a prognosis of disease—free survival for a cancer patient. The invention further relates to methods of prognosticating recurrence or metastasis by determining a level of an MTA1 polypeptide in the patient. Yet further, the methods allow for prognosticating and/or predicting micrometastasis in a patient and offer the improvement in the efficacy of clinical treatment of the patient.

### CROSS-REFERENCE TO RELATED APPLICATIONS

This application claims priority to U.S. Provisional Application No. 60/390,794, which was filed Jun. 21, 2002, and which is hereby incorporated by reference in its entirety.

### STATEMENT REGARDING FEDERALLY SPONSORED RESEARCH OR DEVELOPMENT

The present invention was partially funded by NCI Grant Nos. 3351007302 and 3351007307; therefore, the United States government may have certain rights in this invention.

### TECHNICAL FIELD

The present invention is related to the fields of genetics, molecular biology, cell biology, immunology and medicine. The invention is directed to providing a prognosis of disease-free survival and predicting recurrence, metastasis or micrometastasis in a cancer patient. More particularly, the methods involve determining levels of a MTA1 polypeptide. The invention is useful for cancers involving epithelial carcinomas, particularly epithelial carcinomas for which metastasis is difficult to assess (e.g., breast cancer).

### BACKGROUND OF THE INVENTION

The discovery and clinical validation of markers for cancer of all types which predict prognosis or likelihood of invasive or metastatic spread is one of the major challenges facing oncology. Metastasis is a multistep process by which tumor cells emigrate from the primary tumor, disseminate through blood and lymph vessels, and then are deposited in specific target organs were they re-colonize (Schirrmacher, 1985; Liotta, et al., 1991). For example, in breast cancer, 70% of the approximately 186,000 annual cases present as node negative; however, 30% of these cases will recur after local therapy (i.e., mastectomy) (Boring et al., 1992). Although adjuvant chemotherapy has been demonstrated to improve survival of node negative breast cancer patients, it remains uncertain how to best identify patients whose risk of disease recurrence exceeds their risk of significant therapeutic toxicity (Mansour et al., 1989; Osbourne, 1992).

One of the strongest prognostic factors for cancer-free survival after treatment of the primary tumor is the presence of or absence of local metastatic spread. Several metastasis—associated genes have been identified in efforts to delineate the mechanism by which metastasis occurs and, eventually, may be stopped or prevented. Some of these genes include mst1, nm23, WDNM1, WDNM2, Pgm21, stromelysin-3 and KAI-1 (Dear et al., 1988; Dear et al., 1989; Ebralidze et al., 1989; Bisset et al., 1990; Phillips et al., 1990; Dong et al., 1995; Steeg et al., 1988). Research has shown that each of these genes are associated with progression or metastasis of carcinoma cells, but direct evidence for the specific roles is, for the most part, not available.

Another gene that has been linked to metastatic potential is the metastasis—associated gene, mta1, which was isolated by differential cDNA library screening using the 13762NF rat mammary adenocarcinoma metastatic system (Pencil et al., 1993). The mta1 mRNA was reportedly differentially expressed in highly metastatic rat mammary adenocarcinoma cell lines, but the function of the mta1 gene product was not determined (Toh et al. 1994; Toh et al, 1995). Nawa et al. (2000) described the isolation and analysis of the human homologue (MTA1) cDNA and reported that the MTA1 accumulated in the nucleus. Using antisense phosphorothioate oligonucleotides (PONs). Nawa et al. observed different inconsistent results for the inhibition of cell proliferation in breast carcinoma cell lines. Further, MTA1 is reportedly associated with the nucleosome remodeling histone deacetylase complex (NuRD complex), which is involved in chromatin remodeling (Xue et al., 1998).

Mazumdar et al. (2001) reported that MTA1 binds to the AF-2 region of ER-alpha, that expression is induced by activation of the heregulin/HER2 pathway and that MTA1 is a potent corepressor of ERE transcription, as it blocks the ability of oestradiol to stimulate ER-mediated transcription. Mahoney et al. (2002) reported that (i) forced MTA1 expression enhances migration and invasion of immortalized keratinocytes; (ii) MTA1 expression is necessary but not sufficient for cell survival in the anchorage independent state; (iii) MTA1 contributes to expression of the anti-apoptotic Bcl-2 family member Bcl-X_{L}; and (iv) MTA1 expression in immortalized keratinocytes depends, in part, on activation of the epidermal growth factor receptor (EGFR). Data related to the present invention indicate a more complex relationship between MTA1 protein levels, nodal metastasis, and tumor size. However, these data do indicate a clear association between levels of MTA1 protein and risk of recurrence in the 70% of the approximately 186,000 annual cases who present as node negative disease.

Zhang et al. described a protein that shows significant homology to MTA1, called MTA1-L1, is also a component of the nucleosome remodeling and histone deacetylation complex (Zhang et al., 1998; Zhang et al., 1999; and Futamura et al., 1999). MTA1-L-1 maps to chromosome 11q. A more distantly related MTA1 homologue is MTA2, which maps to chromosome 2 (Nagase, et al., 1999).

Another known prognostic factor for cancer is loss of heterozygosity (LOH). For example, LOH analysis of normal and breast tumor tissues revealed that increasing rates of LOH, which refers to the known loss of large segments or entire chromosomes, were correlated with progressively higher stages of breast cancers (O'Connell et al., 1994; O'Connell et al., 1998). Physical mapping to characterize specific genetic changes in primary breast cancers that result in metastasis was performed and allowed identification of three YAC clones that span the 1.6 Mb metastasis-related region near the D14S62 marker (O'Connell et al., 1999; Martin et al., 2001). Further investigation led to the mapping of MTA1 into the metastasis-related region and established it as a candidate genetic link between LOH, the development of primary cancer and of metastasis.

Prior to the present invention, approaches to provide a prognosis of disease-free survival, recurrence, or the onset of metastasis involved characteristics such as tumor size. For example, in node negative breast cancer evaluation is stratified on the basis of primary tumor size, pathological grade, DNA S-phase fraction (SPF) and steroid hormone receptor status (Allegra et al., 1979; Von Rosen et al., 1989; Fischer et al., 1992; Clark et al., 1994). For example, moderately and well-differentiated tumors <1 cm in size are thought to require only local excision regardless of receptor status, while such tumors from 1 to 3 cm in size that express normal levels of hormone receptor are treated with hormone therapy (Fischer et al., 1993). On the other hand, patients with tumors larger than 2 cm that are poorly differentiated and/or hormone receptor negative are treated with adjuvant chemotherapy (Early Risk Cancer Trialists, 1992, Lancet 339: 1-15; The Ludwig Breast Cancer Study Group (1989). However, therapeutic indications are much less clearly defined for patients having moderately differentiated tumors of 1 to 3 cm in size where the hormone receptor status is borderline or unknown (Gasparini et al., 1993). Deciding the most appropriate therapy for this group of patients, comprising about 70,000 women annually, would benefit from the development of validated prognostic analysis. Similar prognostic tools are needed in most other forms of cancer.

The present invention fulfills a long-sought need in the art by providing methods of prognosticating disease-free survival and of predicting recurrence and the onset of metastasis in a cancer patient. The methods of the present invention involved the discovery by Applicants of the statistical correlation of MTA1 polypeptide levels with recurrence in breast cancer patients. The methods are particularly useful in node negative breast cancer patients in which no clinical evidence of local spread is observed, but about 30% of these patients recur with metastasis. Further, the methods of the present invention are used to identify the high risk and the low risk node negative cancer patients by, in part, predicting micrometastasis, thereby improving the efficacy of treatment.

### BRIEF SUMMARY OF THE INVENTION

An embodiment of the invention is a method of providing a prognosis of disease-free survival in a cancer patient comprising: obtaining a sample from said patient; measuring MTA1 polypeptide level in said sample; and predicting said disease-free survival based upon the measured level of said MTA1 polypeptide, wherein when said level of MTA1 polypeptide level is increased as compared to a normal level, said disease-free survival is decreased. In a specific embodiment, the patient has an epithelial-derived cancer. In other specific embodiments, the cancer is breast, esophagus, colon, gastric, skin, liver, pancreatic, bladder, ovarian, cervical, testicular, brain or lung. In another embodiment of the invention, the sample comprises a fluid, a tissue or a cell.

In a specific embodiment of the invention, the MTA1 polypeptide is SEQ ID NO:1 or a structural variant thereof. In another embodiment of the invention, the level is measured using an anti-MTA1 antibody that selectively cross-reacts with the MTA1 polypeptide, which in further specific embodiments is SEQ ID NO:1 or a structural variant thereof.

In another embodiment of the invention, the anti-MTA1 antibody binds to a specific MTA1 peptide sequence. In a further specific embodiment, the anti-MTA1 antibody binds to SEQ ID NO:6.

An embodiment of the invention is a method of prognosticating metastasis in a cancer patient comprising: obtaining a sample from said patient; measuring MTA1 polypeptide level in the said sample; determining a prognosis based upon the measured level of said MTA1 polypeptide, wherein an increased level of MTA1 polypeptide level as compared to a normal level indicates a poor prognosis.

An embodiment of the invention is a method of prognosticating micrometastasis in a cancer patient comprising: obtaining a sample of said patient; measuring MTA1 polypeptide level in said sample; determining a prognosis based upon the measured level of said MTA1 polypeptide, wherein an increased level of MTA1 polypeptide as compared to a normal level indicates a poor prognosis.

A embodiment of the invention is an anti-MTA1 antibody that selectively cross-reacts with a MTA1 polypeptide comprising SEQ ID NO:1 or a structural variant thereof. In another specific embodiment, the antibody is a polyclonal antibody or a monoclonal antibody.

An embodiment of the invention is a kit comprising an anti-MTA1 antibody in a suitable container, wherein said antibody selectively cross-reacts with an MTA1 polypeptide. In a specific embodiment, the MTA1 polypeptide is SEQ ID NO:1 or a structural variant thereof.

An embodiment of the invention is a method of treating a cancer patient comprising administering to said patient a therapeutically effective amount of a therapeutic agent that decreases a level of a MTA1 polypeptide. In a specific embodiment, the therapeutic agent interferes with translation of the MTA1 polypeptide. In a further specific embodiment, the therapeutic agent comprises a polynucleotide sequence complementary to a specific MTA1 peptide sequence.

In another specific embodiment, the therapeutic agent neutralizes biological activity of the MTA1 polypeptide. In another specific embodiment, the therapeutic agent binds to a specific MTA1 peptide sequence.

An embodiment of the invention is a method of screening for a candidate therapeutic agent that improves disease-free survival in a cancer patient comprising: introducing to a cell a test agent, wherein said cell comprises a polynucleotide encoding a MTA1 polypeptide or a structural variant thereof under control of a promoter which is operable in said cell; and measuring said MTA1 polypeptide or structural variant thereof level after introduction of the test agent, wherein when the measured level is decreased following the introduction, the test agent is the therapeutic agent that improves disease-free survival in said patient. In a specific embodiment, the cell is a cancer cell. In a further specific embodiment, the therapeutic agent is an antagonist of MTA1. In a specific embodiment, the therapeutic agent interferes with translation of the MTA1 polypeptide. In a specific embodiment, the therapeutic agent is an antisense oligonucleotide.

In a further specific embodiment, said antisense oligonucleotide comprises a polynucleotide sequence complementary to a sequence encoding a specific MTA1 polypeptide sequence. In another specific embodiment, the therapeutic agent binds to the MTA1 polypeptide or structural variant, wherein said binding neutralizes the biological activity of the MTA1 polypeptide or structural variant thereof.

### BRIEF DESCRIPTION OF THE DRAWINGS

For a more complete understanding of the present invention, reference is now made to the following descriptions taken in conjunction with the accompanying drawings.

FIG. 1 illustrates the results of MTA1 Western blot analysis on breast tumor lysates as compared to internal controls.

FIGS. 2A to 2D illustrates the results of the MTA1 cell fractionation analysis in breast cancer cell lines, MDA-MB-231 (A); MCF7 (B); MDA-MB-435 (C); and T47D (D).

FIGS. 3A to 3D illustrates the different patterns of MTA1 expression that were observed by immunohistochemical staining.

FIGS. 4A and 4B shows that MTA1 nuclear staining produced a punctate pattern (A) or a diffuse pattern (B).

FIG. 5 illustrates the immunohistochemical scoring system.

FIG. 6 is a graph of the distribution of MTA1 total IHC scores.

FIG. 7 is a Kaplan-Meyer curve correlating MTA1 expression to disease-free survival whereby time indicated is months.

FIG. 8 is a map comparing structural features of the MTA1 polypeptide and the MTA1-L-1 polypeptide.

FIG. 9 illustrates MTA1 expression in normal versus tumor tissue. High MTA1 staining is observed in the nucleus of the invasive breast cancer (IBC), while an adjacent normal terminal ductal lobular unit (TDLU) remains negative for MTA1 expression.

FIG. 10 shows relapse-free survival in untreated node-negative subjects (N=397). Solid lines represent Kaplan-Meier estimates of adjusted relapse-free survival for several values of MTA1. Dotted lines represent Cox regression adjusted estimates of relapse-free survival for values of MTA1, holding tumor size and s phase fraction constant at values equal to the average for untreated cases. P-value refers to the Wald test in Cox regression analysis.

### DETAILED DESCRIPTION OF THE INVENTION

I. Definitions

As used herein the specification, “a” or “an” may mean one or more. As used herein in the claim(s), when used in conjunction with the word “comprising”, the words “a” or “an” may mean one or more than one. As used herein “another” may mean at least a second or more.

The term “antagonist” as used herein is defined as a factor which interferes with, neutralizes or impedes the activity, function or effect of another biological entity. The agent may partially or completely interfere with a biological activity.

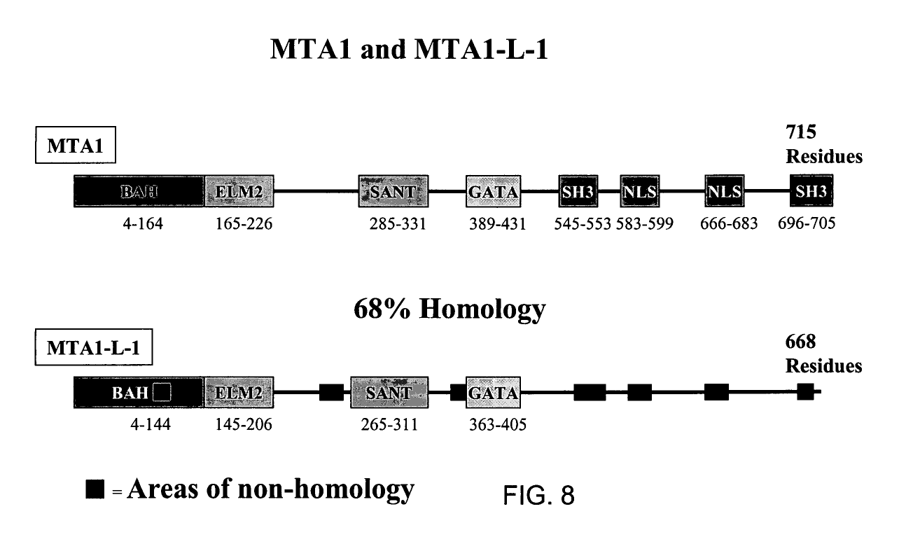

The phrase, “an anti-MTA1 antibody that selectively cross-reacts with an MTA1 polypeptide,” as used herein, refers to an antibody that binds, coordinates, hybridizes, or attaches to the metastasis-associated gene products of chromosome 14q. More specifically, the antibody does not cross-react with MTA homologs, such as MTA1-L-1 and MTA2. For example, non-homologous regions of MTA1-L-1 as compared to MTA1 are indicated in MTA1-L-1 of FIG. 8. Specifically, the peptides of SEQ ID NOS:6 and 7 represent areas of non-homology between the MTA1 protein and the MTA1-L-1 protein. Any peptide directed to non-homologous regions of MTA1 relative to MTA1-L-1, MTA2, or any other related metastasis-associated gene are considered within the scope of the anti-MTA1 antibody that selectively cross-reacts with an MTA1 polypeptide or a structural variant thereof. One skilled in the art recognizes that areas of non-homology are determined using methods well known in the art.

The phrase “candidate therapeutic agent” indicates an agent that has the potential to provide a therapeutic benefit. Such an agent is identified through screening methods known to one with skill in the art.

As used herein, the expressions “cell”, “cell line” and “cell culture” are used interchangeably and all such designations include progeny. Thus, the words “transformants” and “transformed cells” include the primary subject cell and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included. Where distinct designations are intended, it will be clear from the context.

The term “control sample” as used herein indicates a sample that is compared to a patient sample. A control sample may be obtained from a different individual or the same tissue that the patient sample is taken from. However, a noncancerous area may be chosen to reflect the MTA1 polypeptide levels in normal cells for a particular patient. A control may be a cell line, in which serial dilutions are undertaken to determine the exact concentration of elevated MTA1 polypeptide levels. Such levels are compared with a patient sample. A “control sample” may comprise a theoretical patient with an elevated MTA1 polypeptide level that is calculated to be the cutoff point for elevated MTA1 polypeptide levels. A patient sample that has MTA1 polypeptide levels equal to or greater than such a control sample is said to have elevated MTA1 polypeptide levels.

The term “disease-free survival,” as used herein, refers to a period of time beyond the post-diagnosis and/or the initial treatment of primary disease in a patient, that is without recurrence of the disease. For example, a disease-free survival is “low” if the cancer patient demonstrates an increased MTA1 expression level in a tissue.

The term “endocrine therapy” as used herein, is defined as a treatment of or pertaining to any of the ducts or endocrine glands characterized by secreting internally and into the bloodstream from the cells of the gland. For example, endocrine therapy for breast cancer comprises administering tamoxifen, raloxifene, fulvestrant, goserelin acetate, exemestane, megestrol, toremifene, or an aromatase inhibitor, such as anastrozole or letrozole.

The term “epithelial derived,” as used herein, refers to a carcinoma, which is known in the art to refer to a malignant tumor of epithelial origin, of the gastrointestinal tract, breast, bronchus, urothelium, ovary, uterus, cervix, lung, colon, and any other epithelial tissue in the body.

The term “metastasis,” as used herein, refers to the transmission, transformation, or transfer of disease from an original or primary site to one or more sites elsewhere in the body. This secondary spread of disease is a devastating aspect of malignant disease, and a major cause of treatment failures. Patients who have had their primary tumor successfully eradicated are at risk of succumbing, months or years later, to multiple metastases, for which the conventional treatment is systemic chemotherapy.

The term “node-negative,” as used herein, refers to breast cancer that has not infiltrated the lymph nodes of the cancer patient. Currents methods of providing a node-negative diagnosis after surgery at the primary site employ insufficient sensitivities and/or limits of detection, and, consequently, about 30% of node-negative cancer patients experience a recurrence. The recurrence results, in part, as a consequence of the presence of micrometastasis that goes undetected until the level of metastatic cells is above the limit of detection of conventional detection methods. Endocrine therapies (e.g. tamoxifen) are available and routinely prescribed to control breast cancer progression, but only for patients whose breast tumors express the estrogen receptor protein (˜70%). Tamoxifen produces side-effects such as hot flashes, thrombosis, and increased risk of endometrial cancer. Tamoxifen treatment is recommended for only five years, as breast tumors nearly always develop resistance to tamoxifen. When resistance occurs, this anti-cancer drug actually stimulates tumor growth. As a result, for higher risk patients, endocrine therapies such as tamoxifen are used as second line therapies given after standard adjuvant or neoadjuvant chemotherapy (Rivera et al., 2002; and Howell et al., 2001).

The term “node-positive,” as used herein, refers to breast cancer that has infiltrated the lymph nodes of the patient, and the infiltration is detected by conventional means. Generally, this determination is made after surgery at the primary site (i.e., tumor resection), and is a known predictive marker for recurrence and metastasis. It is contemplated that MTA1 levels may be measured in both node-negative and node-positive breast cancers.

The term “polypeptide” as used herein is used interchangeably with the term “protein” and is defined as a molecule which comprises more than one amino acid subunit. An amino acid subunit is discussed in detail in a later section, but includes the natural twenty amino acids, such as phenalanine, valine, aspartic acid, and the like, as well as unnatural or synthetic amino acids. The polypeptide may be an entire protein or it may be a fragment of a protein, such as a peptide or an oligopeptide. The polypeptide may also comprise alterations to the amino acid subunits, such as methylation or acetylation.

The term “prognosis,” as used herein, is defined as a prediction of a probable course and/or outcome of a disease. For example, in the present invention MTA1 is a prognostic marker for recurrence, for metastasis and/or for micrometastasis in a cancer patient.

The term “sample,” as used herein, refers to a biological fluid, a tissue, or a cell that is obtained from a cancer patient. In certain embodiments, the tissue or the cell is obtained from a tumor. In other embodiments, the sample comprises tissue from any site in the body that is at risk of secondary development of cancer.

The phrase “a specific MTA1 peptide sequence,” as used herein, refers to a peptide sequence of the MTA1 polypeptide or a structural variant thereof that is non-homologous to the MTA homologs, including MTA1-L-1 and/or MTA2. In specific embodiments, the MTA1 polypeptide is SEQ ID NO:1 or a structural variant thereof. The peptide sequence is at least about 15 amino acid subunits, and the maximum length is determined by the number of amino acid subunits in the non-homologous region, which is identified as specific to the MTA1 polypeptide. In specific embodiments, the specific MTA1 peptide sequences function as epitopes of metastasis associated gene products of the 14q locus.

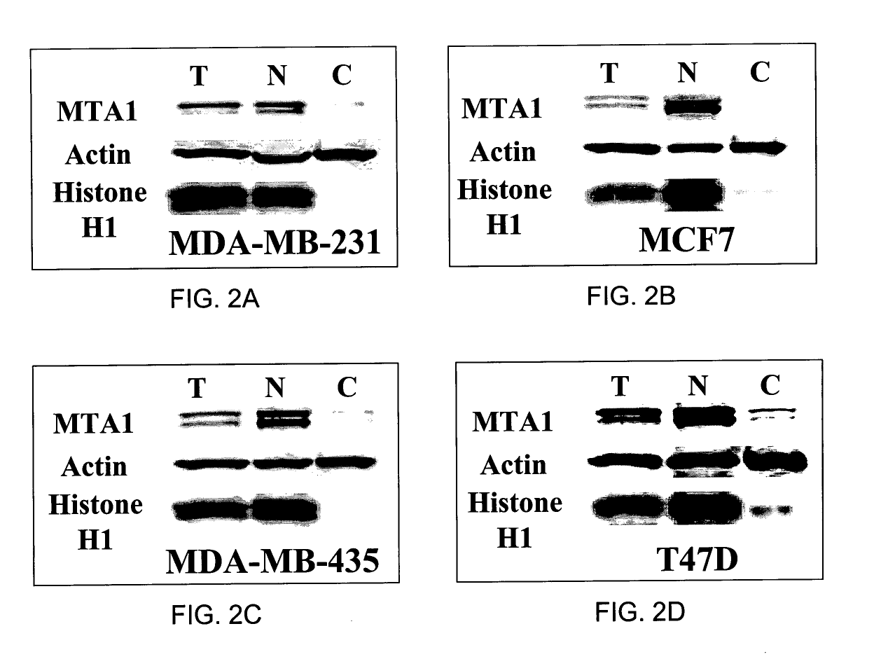

The term “structural variant thereof,” as used herein, refers to an alternative transcript that is generated from the 14q locus in a human. The structural variants encompassed in the present invention are isoforms of the 14q locus, not homologs of MTA1. For example, an alternative form of MTA1 is shown in FIGS. 2A to 2D, which was detected by the specific anti-MTA1 antibody that selectively cross-reacts with a structural variant of SEQ ID NO: 1. Such structural variants of the MTA1 polypeptide are within the scope of functioning as a prognostic and a predictive marker for the methods of the present invention.

The term “therapeutic benefit” as used herein refers to anything that promotes or enhances the well-being of the subject with respect to the medical treatment of his condition, which includes treatment of pre-cancer, cancer, and hyperproliferative diseases. A list of nonexhaustive examples of this includes extension of the subject's life by any period of time, decrease or delay in the neoplastic development of the disease, decrease in hyperproliferation, reduction in tumor growth, delay of metastases, reduction in cancer cell or tumor cell proliferation rate, and a decrease in pain to the subject that can be attributed to the subject's condition. In a specific embodiment, a therapeutic benefit refers to reversing de novo recurrence, metastasis or micrometastasis. In another specific embodiment, a therapeutic benefit refers to preventing the patient from experiencing a recurrence, or developing metastasis or micrometastasis. One with skill in the art realizes that a therapeutic benefit does not necessarily equal a cure, although a cure is an example of a therapeutic benefit.

The term “therapeutically effective amount” as used herein is defined as the amount of a molecule or a compound required to improve a symptom associated with a disease. For example, in the treatment of cancer such as breast cancer, a molecule or a compound which decreases, prevents, delays or arrests any symptom of the breast cancer is therapeutically effective. A therapeutically effective amount of a molecule or a compound is not required to cure a disease but will provide a treatment for a disease. A molecule or a compound is to be administered in a therapeutically effective amount if the amount administered is physiologically significant. A molecule or a compound is physiologically significant if its presence results in technical change in the physiology of a recipient organism.

The term “transcribed” as used herein refers to the generation of a ribonucleic acid from a deoxyribonucleic acid template.

The term “treatment” as used herein is defined as the management of a patient through medical or surgical means. The treatment improves or alleviates at least one symptom of a medical condition or disease and is not required to provide a cure.

It is contemplated that any of the methods described herein may be implemented using compositions of the invention and vice versa. It is further contemplated that any embodiment discussed with respect to an aspect of the invention may be implemented or employed in the context of other aspects of the invention.

II. The Present Invention

The present invention is directed to methods of providing a prognosis of disease-free survival and of disease recurrence in a cancer patient. Generally, the prognosis involves determining an amount of a MTA1 polypeptide that is present in a tissue or cell and is based on Applicants' discovery that MTA1 expression levels are inversely correlated with disease-free survival.

Conventional methods currently used to characterize and sub-classify breast cancer according to prognosis include tumor size, the rate of growth/proliferation of the tumor, status of ERα and PgR expression, and the presence or absence of nodal or regional metastases. The importance of axillary lymph node status as a prognostic factor has long been recognized, with breast cancer patients who present with negative lymph nodes having a 70% likelihood of long-term survival without the clinical appearance of metastases. However, 30% of patients diagnosed with node-negative disease still experience metastatic disease.

The present invention illustrates that MTA1 protein levels are not associated with other known risk factors for recurrence, such as tumor size and nodal metastasis suggesting that MTA1 relates to a unique metastasis mechanism. High MTA1 protein levels predict a high risk of early cancer relapse in untreated, axillary lymph node-negative primary breast cancers. MTA1 protein expression is an independent marker of micrometastatic risk in these otherwise unremarkable primary breast cancers.

MTA1 mRNA levels are elevated in several human cancers, including gastrointestinal, esophageal, pancreatic, lung, and thymoma. In each case, the carcinomas expressed higher levels of MTA1 mRNA than did paired normal tissues, and this overexpression correlated with the invasiveness or metastatic potential of each cancer. Increased MTA1 expression has also been shown in malignant squamous carcinoma cell lines when compared to normal keratinocytes. Recently, Kumar et al. measured MTA1 protein expression using a different antibody in a small subset of node-negative breast cancers, in which they determined that MTA1 expression was higher in tumors than in adjacent normal tissue taken from the same individual.

The association between high MTA1 protein levels and high risk of relapse in node-negative breast cancer is important because current markers of risk of relapse are imperfect. Axillary lymph node status is the single best marker but is determined after surgical node dissection with associated morbidity, and many apparently good prognostic cases eventually relapse. Standard histological evaluation can not exclude the possibility of distant micrometastases. These limitations have led to efforts to identify prognostic biomarkers for patients with node-negative disease. ERα and PgR, two of the most studied biomarkers in breast cancer, predict short-term outcome. Receptor-negative tumors have a higher risk of recurrence for the first several years, but the prognostic significance decreases over the long term.

Due to improved awareness and screening programs, more and more women with primary breast cancer are presenting with node-negative disease. New biomarkers of systemic disease are needed to differentiate metastatic risk in these patients. More accurate indicators of micrometastatic disease would help to select the most appropriate patients for systemic adjuvant treatment. Those with only a low risk would be spared the toxicity and cost of systemic therapy. Our study shows that MTA1 is a significant independent prognostic marker of relapse-free survival (denoting the presence of micrometastases) in node-negative, untreated patients. The lack of a correlation between MTA1 and tumor size or rate of proliferation suggests that this protein may facilitate micrometastatic spread by a different mechanism. MTA1 expression is a particularly valuable in identifying a subset of node-negative patients who have an adverse prognosis. This information can be used to identify those who may need more aggressive treatments.

A. MTA1

MTA1 is a 715 amino acid polypeptide that is associated with the NuRD complex and the metastasis process (Toh et al., 1994; Xue et al., 1998). The human MTA1 is found on chromosome 14q, and is 68% homologous to MTA1-L-1, which is found on chromosome 11q. The human MTA1 polypeptide sequence is found as gene accession number NM_{—}004689 (SEQ ID NO:1) and the human MTA1-L-1 polypeptide sequence is recorded as gene accession number NM_{—}004739 (SEQ ID NO:3). Another known ortholog to MTA1 is MTA2 (gene accession number AF086450: SEQ ID NO:5). The present invention is directed specifically to the expression products of 14q that are involved in metastasis, including the MTA1 polypeptide and structural variants thereof.

In certain embodiments, a sample is obtained from a cancer patient, and the MTA1 polypeptide is measured. The MTA1 polypeptide level is inversely related (inversely correlated) to the disease-free survival in a cancer patient and, thus, the amount of MTA1 polypeptide that is determined provides a prognosis of disease-free survival for the patient.

The sample that is obtained from the cancer patient is a fluid, a tissue or a cell. In certain embodiments, the sample is taken from a tumor or an area that possesses a high metastatic potential, such as a lymph node.

Accurately measuring the metastasis associated gene expression products of 14q is important to the methods of the present invention. The metastasis associated gene expression products of 14q include the MTA1 polypeptide of SEQ ID NO:1 and alternative structural variants, such as the variant shown in FIGS. 2A to 2D.

As provided herein, the MTA1 expression level was determined to function as a strong predictive marker for disease-free survival, and, thus a strong predictive marker for recurrence or for the development of metastasis in a cancer patient. Further, the presence of MTA1 polypeptide indicates the presence of micrometastasis, which is known in the art to contribute to the development of metastasis in clinical diagnosed node negative breast cancers. The use of prognosticating micrometastasis, allows in improved efficacy of treatment of the cancer patient that are not clinically diagnosed as node positive but remain at substantial risk of recurring. For example, the method of indicating the presence of or the probability of developing micrometastasis allows a node-negative breast cancer patient to be identified as at high risk or at low risk for recurrence, and consequently, the accuracy of the clinical treatment is improved.

B. Cancer

It is well established that a variety of cancers are caused, at least in part, by genetic abnormalities that result in either the over-expression of one or more genes, or the expression of an abnormal or mutant gene or genes. Although breast cancer diagnosed in its earliest clinical stages (stage 0, stage Ia) is highly curable, the cure rate for more advanced stages drops precipitously, even after modern combined-modality treatments. Metastatic breast cancer responds to both chemotherapy and hormone therapy, and most patients can be palliated adequately during the 1 to 3 years of usual survival. However, metastatic breast cancer is considered incurable, as demonstrated by the relentless death rates, regardless of the treatment modality utilized. Front-line chemotherapy or hormone therapy programs for correctly selected patients produce objective responses in 50% to 70% of patients, but the median duration of response is usually less than one year. Response rates after second line treatments are considerably lower (20% to 50%), and response durations average 6 months.

Ovarian cancer is also highly curable in its earliest stages, but the overwhelming majority of patients are diagnosed in stages III and IV. Although responsive to chemotherapy, most patients with advanced ovarian cancer relapse and die of their disease. Therefore, improving the efficacy of treatment by prognosticating recurrence, metastasis and/or micrometastasis in these cancer patients has, until the present invention, remained an elusive goal.

The methods of the present invention are directed to breast and ovarian cancers among other epithelial derived carcinomas. More specifically the epithelial-derived cancer that involves the expression of a MTA1 polypeptide is a subject of the present invention.

In specific embodiments, the epithelial-derived cancer (i.e., epithelial-derived carcinoma) of the present invention is found in a tissue of, for example, the breast, esophagus, colon, gastric, skin, liver, pancreas, bladder, ovarian, cervical, uterus, testicular, brain, lung or the like. In further specific embodiments, the cancer is node-negative breast cancer or node-positive breast cancer. These axillary regions are particularly relevant for metastasis and micrometastasis of breast cancer.

In certain embodiments, the patient is untreated following surgery at the primary site. Approximately 60% of persons with cancer undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

Specifically, a breast cancer patient may have undergone a lumpectomy or a mastectomy, although having had a tumor resection is not essential to the operability of the methods of the present invention.

The cancer patient of the present invention may or may not be receiving a therapy such as endocrine therapy, chemotherapy, immunotherapy or gene therapy. Non-limiting examples of endocrine therapies that are contemplated by the present invention include, for example, tamoxifen or raloxifene. Tamoxifen has been the most commonly prescribed drug to treat breast cancer since its approval by the U.S. Food and Drug Administration (FDA) in the 1970s. Tamoxifen is an anti-estrogen and works by competing with the hormone estrogen to bind to estrogen receptors in breast cancer cells. Tamoxifen has been shown to reduce the risk of recurrence of an original cancer and the risk of developing new cancers by working against the effects of estrogen on breast cancer cells. A pharmaceutical composition comprising tamoxifen is generally administered as an oral composition such as a pill or capsule. Raloxifene is another adjuvant employed in endocrine cancers and is an osteoporosis drug that has demonstrated activity in preventing the development of endocrine cancer.

Other adjuvants are well-known in the art, and include complete Freund's adjuvant (a non-specific stimulator of the immune response containing killed Mycobacterium tuberculosis), incomplete Freund's adjuvants, aluminum hydroxide adjuvant, IL-1, IL-2, IL-4, IL-7, IL-12, γ-interferon, GMCSP, BCG, aluminum hydroxide, MDP compounds, such as thur-MDP and nor-MDP, CGP (MTP-PE), lipid A, monophosphoryl lipid A (MPL), RIBI, which contains three components extracted from bacteria, MPL, trehalose dimycolate (TDM) and cell wall skeleton (CWS) in a 2% squalene/Tween 80 emulsion. MHC antigens have also been used as an adjuvant.

In other specific embodiments, the cancer patient is receiving an endocrine therapy comprising goserelin acetate, exemestane, megestrol, toremifene, fulvestrant, a nonsteroidal or a steroidal aromatase inhibitor including, for example, anastrozole and letrozole. More specifically, fulvestrant has demonstrated an ability to destroy estrogen receptors in breast cancer cells, and anastrozole prevents the production of estrogen in the fat and tumor tissue. These therapies are discussed in more detail in the section titled Combination Treatments, provided below.

C. Determining a Protein Level

1. Measuring a Level of a Protein, a Polypeptide or a Peptide

In certain embodiments of the present invention, the metastasis associated gene expression products of chromosome 14q are detected, measured or determined. The expression products include the MTA1 of SEQ ID NO: 1 and structural variants thereof. For purposes of this subsection, the metastasis associated gene expression products of chromosome 14q are referred to collectively as an MTA1 polypeptide. One of ordinary skill in the art is aware of qualitative and quantitative methods and techniques to detect, measure, or determined a polypeptide level.

One specific example of a method to measure an MTA1 polypeptide level involves extracting a frozen tumor sample from a cancer patient and performing a Western blot analysis on the sample. One of ordinary skill in the art is aware of methods well known in the art to perform a Western blot analysis.

In other specific embodiments, a sample obtained from the cancer patient is analyzed by in situ hybridization.

In an exemplary embodiment, the protein or polypeptide levels are detected using antibodies against the MTA1 protein, polypeptides or peptides. The anti-MTA1 antibody selectively cross-reacts with MTA1 or a structural variant thereof and is prepared using peptides having a sequence of a non-homologous region of MTA1 relative to MTA1-L-1, MTA2 or the like. For example, the anti-MTA1 antibody of the present invention comprises SEQ ID NO:6, or other MTA1-specific peptides such as those indicated in FIG. 8 (solid, gray boxes).

In an exemplary embodiment, the agent is a hapten such as multiple antigen peptide (MAP). Methods and systems involving raising antibodies using MAP are known in art, as described by Tam, J. P., 1988; Posnett, et al., 1988; Tam and Zavala, 1989; Tam and Lu, 1989; Lad et al., 1989; Sanatarpia et al., 1988; and Chang et al., 1990, herein incorporated by reference in their entirety.

The antibody of the present invention are monoclonal or polyclonal. In specific embodiments, the antibody is linked to at least one agent to form an antibody conjugate. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule.

Effector molecules comprise molecules having a desired activity, e.g., cytotoxic activity. Non-limiting examples of effector molecules which have been attached to antibodies include toxins, anti-tumor agents, therapeutic enzymes, radio-labeled nucleotides, antiviral agents, chelating agents, cytokines, growth factors, and oligo- or poly-nucleotides. By contrast, a reporter molecule is defined as any moiety which may be detected using an assay. Non-limiting examples of reporter molecules which have been conjugated to antibodies include enzymes, radiolabels, haptens, fluorescent labels, phosphorescent molecules, chemiluminescent molecules, chromophores, luminescent molecules, photoaffinity molecules, colored particles or ligands, such as biotin.

Any antibody of sufficient selectivity, specificity or affinity may be employed as the basis for an antibody conjugate. Such properties may be evaluated using conventional immunological screening methodology known to those of skill in the art. Sites for binding to biological active molecules in the antibody molecule, in addition to the canonical antigen binding sites, include sites that reside in the variable domain. It is known in the art that the variable domain is involved in antibody self-binding (Kang et al., 1988), and contains epitopes (idiotopes) recognized by anti-antibodies (Kohler et al., 1989).

Certain examples of antibody conjugates are those conjugates in which the antibody is linked to a detectable label. “Detectable labels” are compounds and/or elements that can be detected due to their specific functional properties, and/or chemical characteristics, the use of which allows the antibody to which they are attached to be detected, and/or further quantified if desired. Another such example is the formation of a conjugate comprising an antibody linked to a cytotoxic or anti-cellular agent, and may be termed “immunotoxins”.

Antibody conjugates are generally preferred for use as diagnostic agents. Antibody diagnostics generally fall within two classes, those for use in in vitro diagnostics, such as in a variety of immunoassays, and/or those for use in vivo diagnostic protocols, generally known as “antibody-directed imaging”.

Many appropriate imaging agents are known in the art, as are methods for their attachment to antibodies (see, for e.g., U.S. Pat. Nos. 5,021,236; 4,938,948; and 4,472,509, each incorporated herein by reference). The imaging moieties used can be paramagnetic ions; radioactive isotopes; fluorochromes; NMR-detectable substances; X-ray imaging.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and especially bismuth (III).

In the case of radioactive isotopes for therapeutic and/or diagnostic application, one might mention astatine²¹¹, ¹⁴carbon, ⁵¹chromium, ³⁶chlorine, ⁵⁷cobalt, ⁵⁸cobalt, ⁶⁷copper ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium^{99m} and/or yttrium⁹⁰. ¹²⁵I is often being preferred for use in certain embodiments, and technicium^{99m} and/or indium¹¹¹ are also often preferred due to their low energy and suitability for long range detection. Radioactively labeled monoclonal antibodies of the present invention may be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Monoclonal antibodies according to the invention may be labeled with technetium^{99m} by ligand exchange process, for example, by reducing pertechnate with stannous solution, chelating the reduced technetium onto a Sephadex column and applying the antibody to this column. Alternatively, direct labeling techniques may be used, e.g., by incubating pertechnate, a reducing agent such as SNCl₂, a buffer solution such as sodium-potassium phthalate solution, and the antibody. Intermediary functional groups which are often used to bind radioisotopes which exist as metallic ions to antibody are diethylenetriaminepentaacetic acid (DTPA) or ethylene diaminetetracetic acid (EDTA).

Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

Another type of antibody conjugates contemplated in the present invention are those intended primarily for use in vitro, where the antibody is linked to a secondary binding ligand and/or to an enzyme (an enzyme tag) that will generate a colored product upon contact with a chromogenic substrate. Examples of suitable enzymes include urease, alkaline phosphatase, (horseradish) hydrogen peroxidase or glucose oxidase. Preferred secondary binding ligands are biotin and/or avidin and streptavidin compounds. The use of such labels is well known to those of skill in the art and are described, for example, in U.S. Pat. Nos. 3,817,837; 3,850,752; 3,939,350; 3,996,345; 4,277,437; 4,275,149 and 4,366,241; each incorporated herein by reference.

Yet another known method of site-specific attachment of molecules to antibodies comprises the reaction of antibodies with hapten-based affinity labels. Essentially, hapten-based affinity labels react with amino acids in the antigen binding site, thereby destroying this site and blocking specific antigen reaction. However, this may not be advantageous since it results in loss of antigen binding by the antibody conjugate.

Molecules containing azido groups may also be used to form covalent bonds to proteins through reactive nitrene intermediates that are generated by low intensity ultraviolet light (Potter & Haley, 1983). In particular, 2- and 8-azido analogues of purine nucleotides have been used as site-directed photoprobes to identify nucleotide binding proteins in crude cell extracts (Owens & Haley, 1987; Atherton et al., 1985). The 2- and 8-azido nucleotides have also been used to map nucleotide binding domains of purified proteins (Khatoon et al., 1989; King et al., 1989; and Dholakia et al., 1989) and may be used as antibody binding agents.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3α-6α-diphenylglycouril-3 attached to the antibody (U.S. Pat. Nos. 4,472,509 and 4,938,948, each incorporated herein by reference). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl)propionate.

In other embodiments, derivatization of immunoglobulins by selectively introducing sulflhydryl groups in the Fc region of an immunoglobulin, using reaction conditions that do not alter the antibody combining site are contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fc region have also been disclosed in the literature (O'Shannessy et al., 1987). This approach has been reported to produce diagnostically and therapeutically promising antibodies which are currently in clinical evaluation.

a. Antibody Preparation

In certain aspects of the invention, one or more antibodies may be produced to the expressed metastasis associated genes of chromosome 14q, which means the antibodies selectively cross-react with a MTA1 polypeptide of SEQ ID NO:1 or a structural variant thereof. For purposes of this section, the antibodies directed to expressed metastasis associated genes of chromosome 14q are collectively referred to as anti-MTA1 antibodies. These antibodies may be used in various diagnostic or therapeutic applications, described herein below.

Monoclonal antibodies (MAbs) are recognized to have certain advantages, e.g., reproducibility and large-scale production, however their use is not necessary for the operability of the present invention. The invention thus provides monoclonal antibodies of the human, murine, monkey, rat, hamster, rabbit and even chicken origin. Due to the ease of preparation and ready availability of reagents, murine monoclonal antibodies will often be preferred.

Further, “humanized” antibodies are also contemplated, as are chimeric antibodies from mouse, rat, or other species, bearing human constant and/or variable region domains, bispecific antibodies, recombinant and engineered antibodies and fragments thereof.

### i. Polyclonal Antibodies

Polyclonal antibodies to the metastasis associated gene products of chromosome 14q generally are raised in animals by multiple subcutaneous (sc) or intraperitoneal (ip) injections of the metastasis associated gene products of chromosome 14q and an adjuvant. It may be useful to conjugate the metastasis associated gene product of chromosome 14q, or a fragment containing the target amino acid sequence to a protein that is immunogenic in the species to be immunized, e.g. keyhole limpet hemocyanin (KLH), serum albumin, bovine thyroglobulin, or soybean trypsin inhibitor using a bifunctional or derivatizing agent, for example maleimidobenzoyl sulfosuccinimide ester (conjugation through cysteine residues), N-hydroxysuccinimide (through lysine residues), glytaraldehyde, succinic anhydride, SOCl₂, or R¹ N═C═NR, where R and R¹ are different alkyl groups.

Generally, animals are immunized against the immunogenic conjugates or derivatives by combining 1 mg of 1 μg of conjugate (for rabbits or mice, respectively) with 3 volumes of Freud's complete adjuvant and injecting the solution intradermally at multiple sites. One month later the animals are boosted with ⅕ to 1/10 the original amount of conjugate in Freud's complete adjuvant by subcutaneous injection at multiple sites. Seven to 14 days later the animals are bled and the serum is assayed for specific anti-MTA1 antibody titer. Animals are boosted until the titer plateaus. Preferably, the animal boosted with the conjugate of the same metastasis associated gene product of chromosome 14q, but conjugated to a different protein or through a different cross-linking reagent. Conjugates also can be made in recombinant cell culture as protein fusions. Also, aggregating agents such as alum are used to enhance the immune response.

### ii. Monoclonal Antibodies

Monoclonal antibodies are obtained from a population of substantially homogeneous antibodies, i.e., the individual antibodies comprising the population are identical except for possible naturally-occurring mutations that may be present in minor amounts. Thus, the modifier “monoclonal” indicates the character of the antibody as not being a mixture of discrete antibodies. For example, the anti-MTA1 monoclonal antibodies of the invention may be made using the hybridoma method first described by Kohler & Milstein, 1975, or may be made by recombinant DNA methods (Cabilly, et al., U.S. Pat. No. 4,816,567). In the hybridoma method, a mouse or other appropriate host animal, such as hamster is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)). The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high level expression of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 cells available from the American Type Culture Collection, Rockville, Md. USA. Culture medium in which hybridoma cells are growing is assayed for production of monoclonal antibodies directed against a NURR subfamily member or a CRH receptor. Preferably, the binding specificity of monoclonal antibodies produced by hybridoma cells is determined by immunoprecipitation or by an in vitro binding assay, such as radioimmunoassay (RIA) or enzyme-linked immunosorbent assay (ELISA). The binding affinity of the monoclonal antibody can, for example, be determined by the Scatchard analysis of Munson & Pollard, 1980. After hybridoma cells are identified that produce antibodies of the desired specificity, affinity, and activity, the clones may be subcloned by limiting dilution procedures and grown by standard methods. Suitable culture media for this purpose include, for example, Dulbecco's Modified Eagle's Medium or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography. DNA encoding the monoclonal antibodies of the invention is readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences (Morrison, et al., 1984), or by covalently joining to the immunoglobulin coding sequence all or part of the coding sequence for a non-immunoglobulin polypeptide. In that manner, “chimeric” or “hybrid” antibodies are prepared that have the binding specificity of an anti-MTA1 monoclonal antibody herein. Typically such non-immunoglobulin polypeptides are substituted for the constant domains of an antibody of the invention, or they are substituted for the variable domains of one antigen-combining site of an antibody of the invention to create a chimeric bivalent antibody comprising one antigen-combining site having specificity for a NURR subfamily member or a CRH receptor and another antigen-combining site having specificity for a different antigen.

Chimeric or hybrid antibodies also may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate. For diagnostic applications, the antibodies of the invention typically will be labeled with a detectable moiety. The detectable moiety can be any one which is capable of producing, either directly or indirectly, a detectable signal. For example, the detectable moiety may be a radioisotope, such as ³H, ¹⁴C, ³²P, ³⁵S, or ¹²⁵I, a fluorescent or chemiluminescent compound, such as fluorescein isothiocyanate, rhodamine, or luciferin; biotin; radioactive isotopic labels, such as, e.g., 125I, ³²P, ¹⁴C, or ³H, or an enzyme, such as alkaline phosphatase, beta-galactosidase or horseradish peroxidase. Any method known in the art for separately conjugating the antibody to the detectable moiety may be employed, including those methods described by Hunter, et al., 1962; David et al., 1974; Pain, et al., 1981; and Nygren, 1982.

The antibodies of the present invention may be employed in any known assay method, such as competitive binding assays, direct and indirect sandwich assays, and immunoprecipitation assays (Zola, Monoclonal Antibodies: A Manual of Techniques, pp. 147-158 (CRC Press, Inc., 1987). Competitive binding assays rely on the ability of a labeled standard (which may be a specific MTA1 peptide sequence or an immunologically reactive portion thereof) to compete with the test sample analyte (MTA1 polypeptide or structural variant therof) for binding with a limited amount of antibody. The amount of MTA1 polypeptide or structural variant thereof in the test sample is inversely proportional to the amount of standard that becomes bound to the antibodies. To facilitate determining the amount of standard that becomes bound, the antibodies generally are insolubilized before or after the competition, so that the standard and analyte that are bound to the antibodies may conveniently be separated from the standard and analyte which remain unbound. Sandwich assays involve the use of two antibodies, each capable of binding to a different immunogenic portion, or epitope, of the protein to be detected. In a sandwich assay, the test sample analyte is bound by a first antibody which is immobilized on a solid support, and thereafter a second antibody binds to the analyte, thus forming an insoluble three part complex. David & Greene, U.S. Pat. No. 4,376,110. The second antibody may itself be labeled with a detectable moiety (direct sandwich assays) or may be measured using an anti-immunoglobulin antibody that is labeled with a detectable moiety (indirect sandwich assay). For example, one type of sandwich assay is an ELISA assay, in which case the detectable moiety is an enzyme.

### iii. Humanized Antibodies

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers Jones et al., 1986; Riechmann et al., 1988; Verhoeyen et al., 1988, by substituting rodent CDRs or CDR sequences for the corresponding sequences of a human antibody. Accordingly, such “humanized” antibodies are chimeric antibodies (Cabilly, supra), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

It is important that antibodies be humanized with retention of high affinity for the antigen and other favorable biological properties. To achieve this goal, according to a preferred method, humanized antibodies are prepared by a process of analysis of the parental sequences and various conceptual humanized products using three dimensional models of the parental and humanized sequences. Three dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e. the analysis of residues that influence the ability of the candidate immunoglobulin to bind its antigen. In this way, FR residues can be selected and combined from the consensus and import sequence so that the desired antibody characteristic, such as increased affinity for the target antigen(s), is achieved. In general, the CDR residues are directly and most substantially involved in influencing antigen binding. For further details see U.S. Pat. No. 5,821,337 filed Oct. 13, 1998.

### iv. Human Antibodies

Human monoclonal antibodies can be made by the hybridoma method. Human myeloma and mouse-human heteromyeloma cell lines for the production of human monoclonal antibodies have been described, for example, by Kozbor, J. Immunol. (1984), and Brodeur, et al., Monoclonal Antibody Production Techniques and Applications, pp. 51-63 (Marcel Dekker, Inc., New York, 1987). It is also contemplated to produce transgenic animals (e.g. mice) that are capable, upon immunization, of producing a repertoire of human antibodies in the absence of endogenous immunoglobulin production. For example, it has been described that the homozygous deletion of the antibody heavy chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g. Jakobovits et al., 1993. Alternatively, the phage display technology (McCafferty et al., 1990) is used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimicks some of the properties of the B-cell. Phage display can be performed in a variety of formats; for their review see, e.g. Johnson, Kevin S. and Chiswell, David J., Current Opinion in Structural Biology 3, 564-571 (1993). Several sources of V-gene segments can be used for phage display. Clackson et al., 1991 described the isolation of a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., 1991 or Griffith et al., 1993.

In a natural immune response, antibody genes accumulate mutations at a high rate (somatic hypermutation). Some of the changes introduced will confer higher affinity, and B cells displaying high-affinity surface immunoglobulin are preferentially replicated and differentiated during subsequent antigen challenge. This natural process can be mimicked by employing the technique known as “chain shuffling” (Marks et al., 1992). In this method, the affinity of “primary” human antibodies obtained by phage display can be improved by sequentially replacing the heavy and light chain V region genes with repertoires of naturally occurring variants (repertoires) of V domain genes obtained from unimmunized donors. This techniques allows the production of antibodies and antibody fragments with affinities in the nM range. A strategy for making very large phage antibody repertoires (also known as “the mother-of all libraries”) has been described by Waterhouse et al., 1993, and the high affinity human antibody can be directly isolated from the large phage library as previously reported.

Gene shuffling can also be used to derive human antibodies from rodent antibodies, where the human antibody has similar affinities and specificities to the starting rodent antibody. According to this method, which is also referred to as “epitope imprinting”, the heavy or light chain V domain gene of rodent antibodies obtained by phage display technique is replaced with a repertoire of human V domain genes, creating rodent-human chimeras. Selection on antigen results in isolation of human variable capable of restoring a functional antigen-binding site, i.e. the epitope governs (imprints) the choice of partner. When the process is repeated in order to replace the remaining rodent V domain, a human antibody is obtained (see PCT patent application WO 93/06213, published Apr. 1, 1993). Unlike traditional humanization of rodent antibodies by CDR grafting, this technique provides completely human antibodies, which have no framework or CDR residues of rodent origin.

### v. Bispecific Antibodies

Bispecific antibodies are monoclonal, preferably human or humanized, antibodies that have binding specificities for at least two different antigens. In the present case, one of the binding specificities is for a metastasis associated gene product of chromosome 14q, the other one is for any other antigen, and preferably for another receptor or receptor subunit. For example, bispecific antibodies specifically binding a metastasis associated gene product of chromosome 14q and neurotrophic factor, or two different metastasis associated gene product of chromosome 14q are within the scope of the present invention. Methods for making bispecific antibodies are known in the art. Traditionally, the recombinant production of bispecific antibodies is based on the coexpression of two immunoglobulin heavy chain-light chain pairs, where the two heavy chains have different specificities (Millstein and Cuello, 1983). Because of the random assortment of immunoglobulin heavy and light chains, these hybridomas (quadromas) produce a potential mixture of 10 different antibody molecules, of which only one has the correct bispecific structure. The purification of the correct molecule, which is usually done by affinity chromatography steps, is rather cumbersome, and the product yields are low. Similar procedures are disclosed in PCT application publication No. WO 93/08829 (published May 13, 1993), and in Traunecker et al., 1991.

According to a different and more preferred approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2 and CH3 regions. It is preferred to have the first heavy chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are cotransfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance. In a preferred embodiment, of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. For further details of generating bispecific antibodies see, for example, Suresh et al., 1986.

### vi. Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (PCT Application Publication Nos. WO 91/00360 and WO 92/200373; EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

b. Immunodetection Methods

In certain embodiments, the present invention concerns immunodetection methods for binding, purifying, removing, quantifying and/or otherwise generally detecting biological components, such as MTA1 protein components. The anti-MTA1 antibodies prepared in accordance with the present invention may be employed to detect wild-type and/or structural variants of and/or mutant MTA1 proteins, polypeptides and/or peptides. As described throughout the present application, the use of antibodies that selectively cross-react with wild-type, structural variants and/or mutant MTA1 is contemplated. Some immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few. The steps of various useful immunodetection methods have been described in the scientific literature, such as, e.g., Doolittle M H and Ben-Zeev O, 1999; Gulbis B and Galand P, 1993; De Jager R et al., 1993; and Nakamura et al., 1987, each incorporated herein by reference.

In general, the immunobinding methods include obtaining a sample suspected of containing MTA1 protein, polypeptide and/or peptide, and contacting the sample with a first anti-MTA1 antibody in accordance with the present invention, as the case may be, under conditions effective to allow the formation of immunocomplexes.

These methods include methods for purifying wild-type, structural variants, and/or mutant MTA1 proteins, polypeptides and/or peptides as may be employed in purifying wild-type and/or mutant MTA1 proteins, polypeptides and/or peptides from patients' samples and/or for purifying recombinantly expressed wild-type or mutant MTA1 proteins, polypeptides and/or peptides. In these instances, the antibody removes the antigenic wild-type and/or mutant MTA1 protein, polypeptide and/or peptide component from a sample. The antibody will preferably be linked to a solid support, such as in the form of a column matrix, and the sample suspected of containing the wild-type or mutant MTA1 protein antigenic component will be applied to the immobilized antibody. The unwanted components will be washed from the column, leaving the antigen immunocomplexed to the immobilized antibody, which wild-type or mutant MTA1 protein antigen is then collected by removing the wild-type or mutant MTA1 protein and/or peptide from the column.

In other embodiments, the methods of screening for an antagonist of MTA1 that interferes with the binding between the MTA1 and a ligand or a NuRD complex polypeptide comprise fixing a recombinantly expressed wild-type MTA1 proteins, polypeptides and/or peptides to a solid support, such as in the form of a column matrix, in the presence of a candidate substance, and the sample containing the wild-type ligand or NuRD complex protein component will be applied to the immobilized MTA1 polypeptide. The unwanted components will be washed from the column, leaving the NuRD complex protein component uncomplexed to the immobilized MTA1 polypeptide if the candidate substance is a suitable MTA1 antagonist.

The immunobinding methods also include methods for detecting and quantifying the amount of an MTA1 protein reactive component in a sample and the detection and quantification of any immune complexes formed during the binding process. Here, one would obtain a sample suspected of containing an MTA1 protein and/or peptide, and contact the sample with an antibody against MTA1, and then detect and quantify the amount of immune complexes formed under the specific conditions.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to, any MTA1 protein antigens present. After this time, the sample-antibody composition, such as a tissue section, ELISA plate, dot blot or western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. U.S. Patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody and/or a biotin/avidin ligand binding arrangement, as is known in the art.

The anti-MTA1 antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the antibody is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection uses two different antibodies. A first step biotinylated, monoclonal or polyclonal antibody is used to detect the target antigen(s), and a second step antibody is then used to detect the biotin attached to the complexed biotin. In that method the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

The immunodetection methods of the present invention have evident utility in the prognosis of a cancer patient, particularly a patient that presents as node negative, and/or in the screening for a therapeutic agent that decreases a level of the MTA1 polypeptide in the patient. Here, a biological and/or clinical sample suspected of containing a wild-type or mutant MTA1 protein, polypeptide, peptide and/or mutant is used. However, these embodiments also have applications to non-clinical samples, such as in the titering of antigen or antibody samples, for example in the selection of hybridomas.

In the clinical prognosis and/or identification of patients with various forms of cancer, such as estrogen receptor-positive or progesterone receptor-positive cancers, that need endocrine therapy, the detection of MTA1 mutant, and/or an alteration in the levels of MTA1 polypeptide levels, in comparison to the levels in a corresponding biological sample from a normal subject is indicative of a patient with cancer, such as estrogen receptor-positive or progesterone receptor-positive cancers. However, as is known to those of skill in the art, such a clinical prognosis would not necessarily be made on the basis of this method in isolation. Those of skill in the art are very familiar with differentiating between significant differences in types and/or amounts of other biomarkers, which are useful to establish background levels. Indeed, background expression levels are often used to form a “cut-off” above which increased detection will be scored as significant and/or positive.

### i. ELISAs

As detailed above, immunoassays, in their most simple and/or direct sense, are binding assays. Certain preferred immunoassays are the various types of enzyme linked immunosorbent assays (ELISAs) and/or radioimmunoassays (RIA) known in the art. Immunohistochemical detection using tissue sections is also particularly useful. However, it will be readily appreciated that detection is not limited to such techniques, and/or western blotting, dot blotting, FACS analyses, and/or the like may also be used. Irrespective of the format employed, ELISAs have certain features in common, such as coating, incubating and binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

Following all incubation steps in an ELISA, the contacted surface is washed so as to remove non-complexed material. A preferred washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. Preferably, this will be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label. Quantification is then achieved by measuring the degree of color generated, e.g., using a visible spectra spectrophotometer.

### ii. Immunohistochemistry

The antibodies of the present invention may also be used in conjunction with both fresh-frozen and/or formalin-fixed, paraffin-embedded tissue blocks prepared for study by immunohistochemistry (IHC). The method of preparing tissue blocks from these particulate specimens has been successfully used in previous IHC studies of various prognostic factors, and/or is well known to those of skill in the art (Brown et al., 1990; Abbondanzo et al., 1990; Allred et al., 1990).

Briefly, frozen-sections may be prepared by rehydrating 50 ng of frozen “pulverized” tissue at room temperature in phosphate buffered saline (PBS) in small plastic capsules; pelleting the particles by centrifugation; resuspending them in a viscous embedding medium (OCT); inverting the capsule and/or pelleting again by centrifugation; snap-freezing in −70° C. isopentane; cutting the plastic capsule and/or removing the frozen cylinder of tissue; securing the tissue cylinder on a cryostat microtome chuck; and/or cutting 25-50 serial sections.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and/or cutting up to 50 serial permanent sections.

2. Proteinaceous Compositions

In certain embodiments, the present invention concerns at least one proteinaceous molecule, such as a MTA1 polypeptide and/or a structural variant thereof, and/or an anti-MTA1 antibody comprising a specific MTA1 peptide.

As used herein, a “proteinaceous molecule,” “proteinaceous composition,” “proteinaceous compound,” “proteinaceous chain” or “proteinaceous material” generally refers, but is not limited to, a protein or polypeptide of greater than about 200 amino acids or the full length endogenous sequence translated from a gene; a polypeptide of greater than about 100 amino acids; and/or a peptide of from about 3 to about 100 amino acids. All the “proteinaceous” terms described above may be used interchangeably herein.

In certain embodiments, the size of at least one proteinaceous molecule may comprise, but is not limited to, a molecule having about 5 to about 2500 or greater amino molecule residues, and any range derivable therein. The invention includes those lengths of contiguous amino acids of any sequence discussed herein.

As used herein, an “amino molecule” refers to any amino acid, amino acid derivative or amino acid mimic as would be known to one of ordinary skill in the art. In certain embodiments, the residues of the proteinaceous molecule are sequential, without any non-amino molecule interrupting the sequence of amino molecule residues. In other embodiments, the sequence may comprise one or more non-amino molecule moieties. In particular embodiments, the sequence of residues of the proteinaceous molecule may be interrupted by one or more non-amino molecule moieties.

Accordingly, the term “proteinaceous composition” encompasses amino molecule sequences comprising at least one of the 20 common amino acids in naturally synthesized proteins, or at least one modified or unusual amino acid.

In certain embodiments, the proteinaceous composition comprises at least one protein, polypeptide or peptide. For example, in methods that involve an antagonist of an MTA1 polypeptide, the anatgonist may comprise a protein, and as such, a composition comprising the antagonist is a proteinacious composition of the present invention.

Proteinaceous compositions may be made by any technique known to those of skill in the art, including the expression of proteins, polypeptides or peptides through standard molecular biological techniques, the isolation of proteinaceous compounds from natural sources, or the chemical synthesis of proteinaceous materials. The nucleotide and protein, polypeptide and peptide sequences for various genes have been previously disclosed, and may be found at computerized databases known to those of ordinary skill in the art. One such database is the National Center for Biotechnology Information's Genbank and GenPept databases. The coding regions for these known genes may be amplified and/or expressed using the techniques disclosed herein or as would be know to those of ordinary skill in the art. Alternatively, various commercial preparations of proteins, polypeptides and peptides are known to those of skill in the art.

In certain embodiments a proteinaceous compound may be purified. Generally, “purified” will refer to a specific or protein, polypeptide, or peptide composition that has been subjected to fractionation to remove various other proteins, polypeptides, or peptides, and which composition substantially retains its activity, as may be assessed, for example, by the protein assays, as would be known to one of ordinary skill in the art for the specific or desired protein, polypeptide or peptide.

In certain embodiments, the proteinaceous composition may comprise at least a part of an antibody, for example, an antibody against a molecule expressed on a cell's surface, or against an MTA1 polypeptide. As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting. The term “antibody” is also used to refer to any antibody-like molecule that has an antigen binding region, and includes antibody fragments such as Fab′, Fab, F(ab′)₂, single domain antibodies (DABs), Fv, scFv (single chain Fv), and the like. The techniques for preparing, characterizing and using various antibody-based constructs and fragments are well known in the art and are discussed further in an earlier section. (See, e.g., Harlow et al., 1988; incorporated herein by reference).

It is contemplated that virtually any protein, polypeptide or peptide containing component may be used in the compositions and methods disclosed herein. However, it is preferred that the proteinaceous material is biocompatible. In certain embodiments, it is envisioned that the formation of a more viscous composition will be advantageous in that will allow the composition to be more precisely or easily applied to the tissue and to be maintained in contact with the tissue throughout the procedure. In such cases, the use of a peptide composition, or more preferably, a polypeptide or protein composition, is contemplated. Ranges of viscosity include, but are not limited to, about 40 to about 100 poise. In certain aspects, a viscosity of about 80 to about 100 poise is preferred.

a. Functional Aspects

When the present application refers to the function or activity of an MTA1 polypeptide, it is meant that the molecule in question is associated with the NuRD complex and with the metastasis process, and more particularly, is an expression product of chromosome 14q. Determination of which molecules possess this activity may be achieved using assays familiar to those of skill in the art. In certain embodiments, a specific MTA1 peptide of the present invention functions as an antigen that is used to raise an antibody that selectively cross-reacts to the MTA1 polypeptide or structural variants thereof. Determination of which molecules (i.e., peptides) possess this function may be achieved using systems known in the art to identify and determine specific peptide sequences of MTA1.

b. Variants of Proteinaceous Compositions

Amino acid sequence variants of the proteins, polypeptides and peptides of the present invention can be substitutional, insertional or deletion variants. Deletion variants lack one or more residues of the native protein that are not essential for function or immunogenic activity, and are exemplified by variants lacking a transmembrane sequence. Determination of a sequence that functions as a transmembrane sequence is achieved using analytical methods well known to the of skill in the art. Another common type of deletion variant is one lacking secretory signal sequences or signal sequences directing a protein to bind to a particular part of a cell. Insertional mutants typically involve the addition of material at a non-terminal point in the polypeptide. This may include the insertion of an immunoreactive epitope or simply a single residue. Terminal additions, called fusion proteins, are discussed below.

Substitutional variants typically contain the exchange of one amino acid for another at one or more sites within the protein, and may be designed to modulate one or more properties of the polypeptide, such as stability against proteolytic cleavage, without the loss of other functions or properties. Substitutions of this kind preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

The term “biologically functional equivalent” is well understood in the art and is further defined in detail herein. Accordingly, sequences that have between about 70% and about 80%; or more preferably, between about 81% and about 90%; or even more preferably, between about 91% and about 99%; of amino acids that are identical or functionally equivalent to the amino acids of the MTA1 polypeptide provided the biological activity of the protein is maintained. Amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

D. Kits

All the essential materials and/or reagents required for detecting a MTA1 polypeptide or a structural variant thereof in a sample may be assembled together in a kit. This generally comprises an anti-MTA1 antibody to hybridize specifically to individual polypeptides of interest in the practice of the present invention, including SEQ ID NO:1. Also included may be buffers, such as PBS with 10% BSA (bovine serum albumin) and 0.1% NaN₃ to allow for storage of the antibody, stains such as H&E for hisotology analysis, and other reagents suitable for the detection of a metastasis associated gene product of the 14q locus. Such kits generally will comprise, in suitable means, distinct containers for each individual reagent or enzyme as well as for each reaction component.

E. Methods to Treat

The present invention is also directed to treating a cancer patient to improve the disease-free survival of the patient. In certain embodiments of the present invention, there is a method of treating a cancer patient comprising administering to said patient a therapeutically effective amount of a therapeutic agent that decreases a level of a MTA1 polypeptide. By “decrease,” it is meant that the compound functions to interact and interrupt the translation and/or the transcription processes, to suppress a biological activity, to degrade a molecule that is a starting, intermediate or end product of the biosynthetic or metabolic processes of MTA1, to inhibit the metastatic activity, or to compromise the structural integrity of the MTA1 protein molecules throughout the body. It is contemplated that decreasing a level of any of the metastasis associated gene products of the 14q locus affords a therapeutic benefit to the patient. Thus, the term “a MTA1 polypeptide” refers to a metastasis associated gene product of chromosome 14q, which includes the polypeptide comprising SEQ ID NO:1 and structural variants thereof.

By “therapeutic agent,” it is meant a natural or synthetic moleucle, a small molecule of organic or inorganic origin, a natural polymer such as a polynucleotide, a polypeptide, a protein, an enzyme, a carbohydrate, a lipid, or fragments thereof, a vitamin, an antibody, a macromolecule, a synthetic polymer, a radioactive molecule, a chemotherapeutic agent, radiation, a gene therapy, an immunotherapy or the like. The therapeutic agent is administered alone or in combination with any cancer treatment therapy that effects a therapeutic benefit to the patient. These treatments are discussed further below.

In certain embodiments, the therapeutic agent is an anti-MTA1 antibody that selectively cross-reacts with a MTA1 polypeptide. The anti-MTA1 antibody preparation is explained in more detail in the examples and in the above sections. Briefly, the anti-MTA1 antibody is directed to a specific MTA1 peptide sequence and function to neutralize, coordinate, or interfere with the activity of the MTA1 polypeptide by binding thereto. In these embodiments, the specific MTA1 peptide sequence functions as the antigenic determinant or epitope. One skilled in the art is aware that antibody induction does not require biological activity; however, the protein fragment, or oligopeptide must be antigenic. Peptides used to induce specific antibodies may have an amino acid sequence consisting of at least about five amino acids, preferably at least about 10, and more preferably at least about 15. The amino acid sequence used mimics a portion of the amino acid sequence of the natural protein and may contain the entire amino acid sequence of a small, naturally occurring molecule. Procedures well known in the art can be used for the production of antibodies to a MTA1 polypeptide, and these methods are discussed in more detail above.

In other embodiments, the therapeutic agent reduces the MTA1 polypeptide level indirectly by reducing the expression of a MTA1 nucleic acid sequence. Specifically, the therapeutic agent functions to inhibit synthesis of a nucleic acid sequence of SEQ ID NO:2. Alternatively, the transcription of the polynucleotide encoding MTA1 is blocked, and the MTA1 polypeptide level is reduced as a consequence.

In another embodiment, the therapeutic agent functions to reduce the level of a MTA1 polypeptide by inhibiting amino acid synthesis of a sequence comprising SEQ ID NO:1 or a structural variant thereof. This includes not only prevention or cessation of translation of a MTA1 sequence but also includes prevention or cessation of posttranslational processing and/or transport to proper subcellular localization. It is also contemplated that the therapeutic agent functions to increase, accelerate, or promote a MTA1 amino acid breakdown, digestion, catabolism by, for example, modifying the MTA1 amino acid sequence to target the amino acid sequence for degradation, such as ubiquitination.

1. Antisense

In one embodiment, the therapeutic agent interferes with translation of the MTA1 polypeptide, such as administering an antisense oligonucleotide.

An exemplary therapeutic agent that interferes with translation of the MTA1 polypeptide is an antisense oligonucleotide, such as the polynucleotide sequence that encodes the peptide fragment of SEQ ID NO:6. Antisense is known in the art to interrupt the process by which disease-causing proteins are produced, such as the metastasis associated gene products of the 14q locus. Antisense drugs are designed to inhibit the production of disease-causing proteins and have the potential to be more selective and, as a result, more effective and less toxic than traditional drugs. The antisense methods involve messenger RNA (mRNA), which is single-stranded. The mRNA sequence of nucleotides is called “sense” because it results in a gene product (protein). Normally, its unpaired nucleotides are read by transfer RNA anticodons as the ribosome proceeds to translate the message. However, RNA is able to form duplexes with a second strand of RNA having a sequence of bases that is complementary to the first strand. The second strand is called the antisense strand because its sequence of nucleotides is the complement of the message sense. When mRNA forms a duplex with a complementary antisense RNA sequence, translation is blocked. This occurs because i) the ribosome is unable to gain access to the nucleotides in the mRNA, or ii) duplex or double-stranded RNA (dsRNA) is quickly degraded by ribonucleases in the cell. The ability of dsRNA to suppress the expression of a gene corresponding to its own sequence is called RNA interference (RNAi) or post-transcriptional gene silencing (PTGS). With recombinant DNA methods, synthetic genes (DNA) encoding antisense RNA molecules are introduced into a host organism.

Thus, in specific embodiments that involve antisense, a polynucleotide comprises an antisense RNA molecule having a sequence that encodes an MTA1 peptide sequence. In these embodiments, the antisense oligonucleotide targets any portion of the MTA1 mRNA. In a further specific embodiment, the antisense RNA molecule has a sequence that targets a specific MTA1 peptide sequence, however, this specificity is not necessary for operability.

Furthermore, the skilled artisan recognizes that modifications of gene expression can be obtained by designing antisense molecules to the control regions of a MTA1 nucleic acid sequence, i.e. the promoters, enhancers, and introns. Oligonucleotides derived from the transcription initiation site, e.g. between −10 and +10 regions of the leader sequence, are preferred. The antisense molecules may also be designed to block translation of mRNA by preventing the transcript from binding to ribosomes. Similarly, inhibition can be achieved by using “triple helix” base-pairing methodology. Triple helix pairing compromises the ability of the double helix to open sufficiently for the binding of polymerases, transcription factors, or regulatory molecules.

In an alternative embodiment, the therapeutic agent is a ribozyme, which are enzymatic RNA molecules capable of catalyzing the specific cleavage of RNA. The mechanism of ribozyme action involves sequence-specific hybridization of the ribozyme molecule to complementary target RNA, followed by endonucleolytic cleavage. Within the scope of the invention are engineered hammerhead motif ribozyme molecules that can specifically and efficiently catalyze the endonucleolytic cleavage of sequences encoding an MTA1 polypeptide or structural variant thereof. In another embodiment, the ribozyme is a Tetrahymena-type ribozyme.

Antisense molecules and ribozymes of the invention may be prepared by any method known in the art for the synthesis of RNA molecules, including techniques for chemically synthesizing oligonucleotides, and exemplary methods are discussed in a later section. Alternatively, RNA molecules may be generated by in vitro and in vivo transcription of DNA sequences encoding a MTA1 polypeptide. Such DNA sequences may be incorporated into a wide variety of vectors with suitable RNA polymerase promoters such as T7 or SP6. Alternatively, antisense cDNA constructs that synthesize antisense RNA constitutively or inducibly can be introduced into cell lines, cells or tissues.

2. Therapeutic Agents

In certain embodiments, an antagonist of MTA1 is identified for the purpose of treating a cancer patient and functions to improve disease-free survival in the cancer patient.

An exemplary embodiment of a method of screening for the antagonist comprises: (a) introducing to a cell a test agent, wherein the cell comprises a polynucleotide sequence encoding a MTA1 polypeptide under control of a promoter operable in said cell; and (b) measuring the MTA1 polypeptide level, wherein when the measured level is decreased following the introduction, the test agent is the antagonist that improves disease-free survival. In specific embodiments, the cell is a cancer cell. In other specific embodiments, the cell is a recombinant cell in which the polynucleotide encoding the MTA1 polypeptide further comprises an expression vector.

In certain embodiments, the polynucleotide sequence encoding a MTA1 polypeptide comprises SEQ ID NO:2. In specific embodiments, the polynucleotide sequence further comprises a selectable marker, wherein the level of the selectable marker is measured. As one of skill in the art is aware, using selectable markers facilitate analysis in certain cases because the marker may be more readily detected and/or provide a higher throughput assay.

a. Screening for Modulators of Protein Function

The present invention comprises methods for identifying modulators of the function of an MTA1 polypeptide. For purposes of this section, the MTA1 polypeptide includes the expression products of locus 14q that are associated with metastasis. These assays may comprise random screening of large libraries of candidate substances; alternatively, the assays may be used to focus on particular classes of compounds selected with an eye towards structural attributes that are believed to make them more likely to modulate the function of an MTA1 polypeptide.

By function, it is meant that one may assay for an substance or molecule that interferes with translation of the MTA1 polypeptide, that interferes with an interaction between the MTA1 polypeptide and a NuRD complex component or that neutralizes through binding the MTA1 polypeptide. In such embodiments, the modulator compound may interact, bind, or corrdinate with at least one amino acid that is involved in the interaction between MTA1 and the NuRD complex.

To identify an MTA1 polypeptide modulator, one generally will determine the function of the MTA1 polypeptide in the presence and absence of the candidate substance, a modulator defined as any substance that alters function. For example, a method generally comprises: providing a candidate modulator; admixing the candidate modulator with an isolated compound or cell, or a suitable experimental animal; measuring one or more characteristics of the compound, cell or animal; and comparing the characteristic measured with the characteristic of the compound, cell or animal in the absence of said candidate modulator, wherein a difference between the measured characteristics indicates that said candidate modulator is, indeed, a modulator of the compound, cell or animal. Assays may be conducted in cell free systems, in isolated cells, or in organisms including transgenic animals.

It will, of course, be understood that all the screening methods of the present invention are useful in themselves notwithstanding the fact that effective candidates may not be found. The invention provides methods for screening for such candidates, not solely methods of finding them.

### i. Modulators

As used herein the term “candidate substance” refers to any molecule that may potentially inhibit a MTA1 polypeptide activity. The candidate substance may be a protein or fragment thereof, a small molecule, or even a nucleic acid molecule. It may prove to be the case that the most useful pharmacological compounds will be compounds that are structurally related to an estrogen receptor or derivative thereof. Using lead compounds to help develop improved compounds is know as “rational drug design” and includes not only comparisons with know inhibitors and activators, but predictions relating to the structure of target molecules.

The goal of rational drug design is to produce structural analogs of biologically active polypeptides or target compounds. By creating such analogs, it is possible to fashion drugs, which are more active or stable than the natural molecules, which have different susceptibility to alteration or which may affect the function of various other molecules. In one approach, one would generate a three-dimensional structure for a target molecule, or a fragment thereof. This could be accomplished by x-ray crystallography, computer modeling or by a combination of both approaches.

It also is possible to use antibodies to ascertain the structure of a target compound activator or inhibitor, such as the anti-MTA1 antibodies provided herein. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include fragments or parts of naturally-occurring compounds, or may be found as active combinations of known compounds, which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be peptide, polypeptide, polynucleotide, small molecule inhibitors or any other compounds that may be designed through rational drug design starting from known inhibitors or stimulators.

Other suitable modulators include antisense molecules, ribozymes, and antibodies (including single chain antibodies), each of which would be specific for the target molecule. Such compounds are described in greater detail elsewhere in this document. For example, an antisense molecule that bound to a translational or transcriptional start site, or splice junctions, would be an ideal candidate inhibitor.

In addition to the modulating compounds initially identified, the inventors also contemplate that other sterically similar compounds may be formulated to mimic the key portions of the structure of the modulators. Such compounds, which may include peptidomimetics of peptide modulators, may be used in the same manner as the initial modulators. In other embodiments, the sterically similar compound is a hapten or a small organic molecule that interacts, binds, corrdinates or associates with an amino acid that is considered critical for function.

An inhibitor according to the present invention may be one which exerts its inhibitory effect upstream, downstream or directly on an MTA1 polypeptide. Regardless of the type of antagonist identified by the present screening methods, the effect of the inhibition by such a compound results in reduced MTA1 polypeptide activity and/or a reduced MTA1 polypeptide level as compared to that observed in the absence of the added candidate substance.

### ii. In Vitro Assays

A quick, inexpensive and easy assay to run is an in vitro assay. Such assays generally use isolated molecules, can be run quickly and in large numbers, thereby increasing the amount of information obtainable in a short period of time. A variety of vessels may be used to run the assays, including test tubes, plates, dishes and other surfaces such as dipsticks or beads.

One example of a cell free assay is a binding assay. While not directly addressing function, the ability of a modulator to bind to a target molecule in a specific fashion is strong evidence of a related biological effect. For example, binding of a molecule to a target may, in and of itself, be inhibitory, due to steric, allosteric or charge-charge interactions. The target may be either free in solution, fixed to a support, expressed in or on the surface of a cell. Either the target or the compound may be labeled, thereby permitting determining of binding. Usually, the target will be the labeled species, decreasing the chance that the labeling will interfere with or enhance binding. Competitive binding formats can be performed in which one of the agents is labeled, and one may measure the amount of free label versus bound label to determine the effect on binding.

A technique for high throughput screening of compounds is described in WO 84/03564. Large numbers of small peptide test compounds are synthesized on a solid substrate, such as plastic pins or some other surface. Bound polypeptide is detected by various methods.

### iii. In Cyto Assays

The present invention also contemplates the screening of compounds for their ability to modulate MTA1 polypeptide levels or activity in cells. Various cell lines can be utilized for such screening assays, including cells specifically engineered for this purpose. Depending on the assay, culture may be required. The cell is examined using any of a number of different physiologic assays. Alternatively, molecular analysis may be performed, for example, looking at protein expression, mRNA expression (including differential display of whole cell or polyA RNA) and others.

### iv. In Vivo Assays

In vivo assays involve the use of various animal models, including transgenic animals that have been engineered to have specific defects, or carry markers that can be used to measure the ability of a candidate substance to reach and effect different cells within the organism. Due to their size, ease of handling, and information on their physiology and genetic make-up, mice are a preferred embodiment, especially for transgenics. However, other animals are suitable as well, including rats, rabbits, hamsters, guinea pigs, gerbils, woodchucks, cats, dogs, sheep, goats, pigs, cows, horses and monkeys (including chimps, gibbons and baboons). Assays for modulators may be conducted using an animal model derived from any of these species.

In such assays, one or more candidate substances are administered to an animal, and the ability of the candidate substance(s) to alter one or more characteristics, as compared to a similar animal not treated with the candidate substance(s), identifies a modulator. The characteristics may be any of those discussed above with regard to the function of a particular compound (e.g., enzyme, receptor, hormone) or cell (e.g., growth, tumorigenicity, survival), or instead a broader indication such as behavior, anemia, immune response, etc.

Thus, the present invention provides methods of screening for a candidate substance that functions as an antagonist of an MTA1 polypeptide. In these embodiments, the present invention is directed to a method for determining the ability of a candidate substance to function as an antagonist of an MTA1 polypeptide, generally including the steps of: administering a candidate substance to the animal; and determining the ability of the candidate substance to reduce one or more functional characteristics of an MTA1 polypeptide, including, for example, the inhibition of cellular hyperproliferation.

Treatment of these animals with test compounds will involve the administration of the compound, in an appropriate form, to the animal. Administration will be by any route that could be utilized for clinical or non-clinical purposes, including but not limited to oral, nasal, buccal, or even topical. Alternatively, administration may be by intratracheal instillation, bronchial instillation, intradermal, subcutaneous, intramuscular, intraperitoneal or intravenous injection. Specifically contemplated routes are systemic intravenous injection, regional administration via blood or lymph supply, or directly to an affected site.

Determining the effectiveness of a compound in vivo may involve a variety of different criteria. Also, measuring toxicity and dose response can be performed in animals in a more meaningful fashion than in in vitro or in cyto assays.

b. Nucleic Acid Sequences

In certain embodiments, the invention concerns the use of MTA1 nucleic acids, genes and gene products, such as the human MTA1 polynucleotide sequence of SEQ ID NO:2 or fragments thereof, and those polynucleotide sequences encoding SEQ ID NOS:6 and 7.

Certain aspects of the present invention concern at least one MTA1 nucleic acid sequence. In certain aspects, the MTA1 nucleic acid comprises a wild-type MTA1 nucleic acid. In particular aspects, the MTA1 nucleic acid encodes for at least one transcribed nucleic acid. In particular aspects, the MTA1 nucleic acid encodes at least one MTA1 protein, polypeptide or peptide, or structural variant thereof. In other aspects, the MTA1 nucleic acid comprises at least one nucleic acid segment of SEQ ID NO:2 functional equivalent thereof.

C. Selectable and Screenable Markers

In certain embodiments of the invention, the cells contain nucleic acid constructs of the present invention, a cell may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

d. Host Cells

In certain embodiments of the present invention, in particularly those directed to screening for an antagonist of MTA1, a host cell is involved. As used herein, the terms “cell,” “cell line,” and “cell culture” also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organisms that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny.

Host cells may be derived from prokaryotes or eukaryotes, depending upon whether the desired result is replication of the vector or expression of part or all of the vector-encoded nucleic acid sequences. Numerous cell lines and cultures are available for use as a host cell, and they can be obtained through the American Type Culture Collection (ATCC), which is an organization that serves as an archive for living cultures and genetic materials, which is readily accessible on the world wide web. An appropriate host can be determined by one of skill in the art based on the vector backbone and the desired result. A plasmid or cosmid, for example, can be introduced into a prokaryote host cell for replication of many vectors. Bacterial cells used as host cells for vector replication and/or expression include DH5α, JM109, and KC8, as well as a number of commercially available bacterial hosts such as SURE® Competent Cells and SOLOPACK™ Gold Cells (STRATAGENE®, La Jolla). Alternatively, bacterial cells such as E. coli LE392 could be used as host cells for phage viruses.

Examples of eukaryotic host cells for replication and/or expression of a vector include HeLa, NIH3T3, Jurkat, 293, Cos, CHO, Saos, and PC12. Further, non-limiting examples of mammalian host cell lines include Vero and HeLa cells, other B- and T-cell lines, such as CEM, 721.221, H9, Jurkat, Raji, etc., as well as cell lines of Chinese hamster ovary, W138, BHK, COS-7, 293, HepG2, 3T3, RIN and MDCK cells. In addition, a host cell strain may be chosen that modulates the expression of the inserted sequences, or that modifies and processes the gene product in the manner desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to insure the correct modification and processing of the foreign protein expressed.

Animal cells can be propagated in vitro in two modes: as non-anchorage-dependent cells growing in suspension throughout the bulk of the culture or as anchorage-dependent cells requiring attachment to a solid substrate for their propagation (i.e., a monolayer type of cell growth). Non-anchorage dependent or suspension cultures from continuous established cell lines are the most widely used means of large-scale production of cells and cell products. However, suspension cultured cells have limitations, such as tumorigenic potential and lower protein production than adherent cells.

Similarly, a viral vector may be used in conjunction with either a eukaryotic or prokaryotic host cell, particularly one that is permissive for replication or expression of the vector.

Following transduction with an expression construct or vector according to some embodiments of the present invention, primary mammalian cell cultures may be prepared in various ways. In order for the cells to be kept viable while in vitro and in contact with the expression construct, it is necessary to ensure that the cells maintain contact with the correct ratio of oxygen and carbon dioxide and nutrients but are protected from microbial contamination. Cell culture techniques are well documented and are disclosed herein by reference (Freshner, 1992).

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

A number of selection systems may be used including, but not limited to, HSV thymidine kinase, hypoxanthine-guanine phosphoribosyltransferase and adenine phosphoribosyltransferase genes, in tk-, hgprt- or aprt-cells, respectively. Also, anti-metabolite resistance can be used as the basis of selection: for dhfr, which confers resistance to; gpt, which confers resistance to mycophenolic acid; neo, which confers resistance to the aminoglycoside G418; and hygro, which confers resistance to hygromycin.

e. Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986 and 4,879,236, both herein incorporated by reference, and which can be bought, for example, under the name MAXBAC® 2.0 from INVITROGEN® and BACPACK™ BACULOVIRUS EXPRESSION SYSTEM FROM CLONTECH®. Other examples of a bacterial expression systems are AFFINITY® T7 RNA polymerase-based pCAL vectors express cloned proteins as fusions with the calmodulin-binding peptide (CBP) tag and CLONTECH®'s HAT Protein Expression System.

Other examples of expression systems include STRATAGENE®'s COMPLETE CONTROL™ Inducible Mammalian Expression System, which involves a synthetic ecdysone-inducible receptor. Also from STRATAGENE® is the pET E. COLI EXPRESSION SYSTEM is a widely used in vivo bacterial expression system due to the strong selectivity of the bacteriophage T7 RNA polymerase, the high level of activity of the polymerase and the high efficiency of translation. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

Other assays may be used to identify responsive elements in a promoter region or gene. Such assays will be known to those of skill in the art (see for example, Sambrook et al., 1989; Zhang et al, 1997; Shan et al., 1997; Dai and Burnstein, 1996; Cleutjens et al., 1997; Ng et al., 1994; Shida et al., 1993), and include DNase I footprinting studies, Elecromobility Shift Assay patterns (EMSA), the binding pattern of purified transcription factors, effects of specific transcription factor antibodies in inhibiting the binding of a transcription factor to a putative responsive element, Western analysis, nuclear run-on assays, and DNA methylation interference analysis.

Gene expression may be determined by measuring the production of RNA, protein or both. The gene product (RNA or protein) may be isolated and/or detected by methods well known in the art. Following detection, one may compare the results seen in a given cell line or individual with a statistically significant reference group of non-transformed control cells. Alternatively, one may compare production of RNA or protein products in cell lines transformed with the same gene operably linked to various mutants of a promoter sequence. In this way, it is possible to identify regulatory regions within a novel promoter sequence by their effect on the expression of an operably linked gene.

f. Non-protein Expressing Sequences

DNA may be introduced into a host cell for the purpose of expressing RNA transcripts that function to affect a phenotype. Two examples are antisense RNA and RNA with ribozyme activity. As discussed in earlier in this section, both may serve possible functions in reducing or eliminating expression of native or introduced genes.

Genes may be constructed or isolated, which when transcribed, produce antisense RNA that is complementary to all or part(s) of a targeted messenger RNA(s). As discussed earlier, the antisense RNA reduces production of the polypeptide product of the messenger RNA. The polypeptide product may be any protein encoded by the host's genome. The aforementioned genes will be referred to as antisense genes. An antisense gene may thus be introduced into a host cell by transformation methods known to a skilled artisan (i.e., electroporation) to reduce expression of a selected protein of interest. Reduction of the enzyme activity may reduce or eliminate products of the reaction which include any enzymatically synthesized compound in the host such as fatty acids, amino acids, carbohydrates, nucleic acids and the like. Alternatively, the protein may be a storage protein, or a structural protein, the decreased expression of which may lead to changes in amino acid composition or morphological changes respectively. The possibilities cited above are provided only by way of example and do not represent the full range of applications.

3. Nucleic Acid Delivery

The general approach to the aspects of the present invention concerning compositions and/or therapeutics is to provide a cell with a gene construct encoding a specific and/or desired protein, polypeptide and peptide, thereby permitting the desired activity of the proteins to take effect. While it is conceivable that the gene construct and/or protein may be delivered directly, a preferred embodiment involves providing a nucleic acid encoding a specific and desired protein, polypeptide and peptide to the cell. Following this provision, the proteinaceous composition is synthesized by the transcriptional and translational machinery of the cell, as well as any that may be provided by the expression construct. In providing antisense, ribozymes and other inhibitors, the preferred mode is also to provide a nucleic acid encoding the construct to the cell.

In certain embodiments of the invention, the nucleic acid encoding the gene may be stably integrated into the genome of the cell. In yet further embodiments, the nucleic acid may be stably maintained in the cell as a separate, episomal segment of DNA. Such nucleic acid segments and “episomes” encode sequences sufficient to permit maintenance and replication independent of and in synchronization with the host cell cycle. How the expression construct is delivered to a cell and/or where in the cell the nucleic acid remains is dependent on the type of expression construct employed.

Delivery systems for tranfection of nucleic acids into cells may utilize either viral or non-viral methods. A targeted system for non-viral forms of DNA or RNA requires four components: 1) the DNA or RNA of interest; 2) a moiety that recognizes and binds to a cell surface receptor or antigen; 3) a DNA binding moiety; and 4) a lytic moiety that enables the transport of the complex from the cell surface to the cytoplasm. Further, liposomes and cationic lipids can be used to deliver the therapeutic gene combinations to achieve the same effect. Potential viral vectors include expression vectors derived from viruses such as adenovirus, vaccinia virus, herpes virus, and bovine papilloma virus. In addition, episomal vectors may be employed. Other DNA vectors and transporter systems are known in the art.

One skilled in the art recognizes that expression vectors derived from retroviruses, adenovirus, herpes or vaccinia viruses, or from various bacterial plasmids, may be used for delivery of nucleotides sequences to a targeted organ, tissue or cell population. Methods which are well known to those skilled in the art can be used to construct recombinant vectors which will express antisense nucleotides of the gene encoding a MTA1 polypeptide. The genes can be turned off by transfecting a cell or tissue with expression vectors which express high levels of a desired gene-encoding fragment. Such constructs can flood cells with untranslatable sense or antisense sequences. Even in the absence of integration into the DNA, such vectors may continue to transcribe RNA molecules until all copies are disabled by endogenous nucleases. Transient expression may last for a month or more with a non-replicating vector and even longer if appropriate replication elements are a part of the vector system.

In a specific embodiment, the transfection of nucleic acid is facilitated by a transport protein, as described in Subramanian et al. (1999). Briefly, a peptide M9 is chemically bound to a cationic peptide as a carrier molecule. The cationic complex binds the negatively charged nucleic acid of interest, followed by binding of M9 to a nuclear transport protein, such as transportin.

Once the expression construct has been delivered into the cell the nucleic acid encoding the gene of interest may be positioned and expressed at different sites. In certain embodiments, the nucleic acid encoding the gene may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In one embodiment of the invention, the expression construct may simply consist of naked recombinant DNA or plasmids. Transfer of the construct may be performed by any of the methods mentioned above which physically or chemically permeabilize the cell membrane. This is particularly applicable for transfer permeabilize the cell membrane. This is particularly applicable for transfer in vitro but it may be applied to in vivo use as well. Dubensky et al. (1984) successfully injected polyomavirus DNA in the form of CaPO₄ precipitates into liver and spleen of adult and newborn mice demonstrating active viral replication and acute infection. Benvenisty and Neshif (1986) also demonstrated that direct intraperitoneal injection of CaPO₄ precipitated plasmids results in expression of the transfected genes. It is envisioned that DNA encoding a gene of interest may also be transferred in a similar manner in vivo and express the gene product.

Another embodiment of the invention for transferring a naked DNA expression construct into cells may involve particle bombardment. This method depends on the ability to accelerate DNA coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

Selected organs including the liver, skin, and muscle tissue of rats and mice have been bombarded in vivo (Yang et al, 1990; Zelenin et al., 1991). This may require surgical exposure of the tissue or cells, to eliminate any intervening tissue between the gun and the target organ, i.e., ex vivo treatment. Again, DNA encoding a particular gene may be delivered via this method and still be incorporated by the present invention.

Other expression constructs which can be employed to deliver a nucleic acid encoding a particular gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific.

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987) and transferrin (Wagner et al., 1990). A synthetic neoglycoprotein, which recognizes the same receptor as ASOR, has been used as a gene delivery vehicle (Ferkol et al., 1993; Perales et al., 1994) and epidermal growth factor (EGF) has also been used to deliver genes to squamous carcinoma cells (Myers, EPO 0273085).

In other embodiments, the delivery vehicle may comprise a ligand and a liposome. For example, Nicolau et al. (1987) employed lactosyl-ceramide, a galactose-terminal asialganglioside, incorporated into liposomes and observed an increase in the uptake of the insulin gene by hepatocytes. Thus, it is feasible that a nucleic acid encoding a particular gene also may be specifically delivered into a cell type such as lung, epithelial or tumor cells, by any number of receptor-ligand systems with or without liposomes. For example, epidermal growth factor (EGF) may be used as the receptor for mediated delivery of a nucleic acid encoding a gene in many tumor cells that exhibit upregulation of EGF receptor. Mannose can be used to target the mannose receptor on liver cells. Also, antibodies to CD5 (CLL), CD22 (lymphoma), CD25 (T-cell leukemia) and MAA (melanoma) can similarly be used as targeting moieties.

In certain embodiments, gene transfer may more easily be performed under ex vivo conditions. Ex vivo gene therapy refers to the isolation of cells from an animal, the delivery of a nucleic acid into the cells, in vitro, and then the return of the modified cells back into an animal. This may involve the surgical removal of tissue/organs from an animal or the primary culture of cells and tissues. Anderson et al., U.S. Pat. No. 5,399,346, and incorporated herein in its entirety, disclose ex vivo therapeutic methods.

4. Pharmaceutical Preparations

Pharmaceutical compositions of the present invention comprise an effective amount of one or more forms of therapeutic compound that decreases an MTA1 polypeptide level and additional agent dissolved or dispersed in a pharmaceutically acceptable carrier or excipient. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains at least one therapeutic compound that reduces a level of the MTA1 polypeptide or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The therapeutic compound of the present invention may comprise different types of carriers depending on whether it is to be administered in solid, liquid or aerosol form, and whether it need to be sterile for such routes of administration as injection. The present invention can be administered intravenously, intradermally, intraarterially, intraperitoneally, intralesionally, intracranially, intraarticularly, intraprostaticaly, intrapleurally, intratracheally, intranasally, intravitreally, intravaginally, rectally, topically, intratumorally, intramuscularly, intraperitoneally, subcutaneously, intravesicularlly, mucosally, intrapericardially, orally, topically, locally, using aerosol, injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 1990, incorporated herein by reference).

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The therapeutic compound may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

In other embodiments, one may use eye drops, nasal solutions or sprays, aerosols or inhalants in the present invention. Such compositions are generally designed to be compatible with the target tissue type. In a non-limiting example, nasal solutions are usually aqueous solutions designed to be administered to the nasal passages in drops or sprays. Nasal solutions are prepared so that they are similar in many respects to nasal secretions, so that normal ciliary action is maintained. Thus, in preferred embodiments the aqueous nasal solutions usually are isotonic or slightly buffered to maintain a pH of about 5.5 to about 6.5. In addition, antimicrobial preservatives, similar to those used in ophthalmic preparations, drugs, or appropriate drug stabilizers, if required, may be included in the formulation. For example, various commercial nasal preparations are known and include drugs such as antibiotics or antihistamines.

In certain embodiments, the therapeutic compound that decreases an MTA1 polypeptide level is prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. Preferred carriers for oral administration comprise inert diluents, assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof.

In certain preferred embodiments an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

Additional formulations which are suitable for other modes of administration include suppositories. Suppositories are solid dosage forms of various weights and shapes, usually medicated, for insertion into the rectum, vagina or urethra. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

In particular embodiments, prolonged absorption of an injectable composition can be brought about by the use in the compositions of agents delaying absorption, such as, for example, aluminum monostearate, gelatin or combinations thereof.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

### EXAMPLES

The following examples are included to demonstrate preferred embodiments of the invention. It should be appreciated by those skilled in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1

### Method of Constructing an Anti-MTA1 antibody

Peptide antigen selection software was used to identify optimum MTA1-specific peptide sequences in regions of non-homology as compared to the closely related MTA-L1 peptide, which has 68% sequence homology (FIG. 8). A list of peptides was developed as candidates, based in part on antigenicity, for the production of a specific anti-MTA1 antibody, which does not cross-react with MTA homologs. Two of the candidate peptides were chosen for further characterization: i) N-terminal amino acids 88-104, ENPEMVDLPEKLKHQLR (SEQ ID NO:6); and ii) C-terminal amino acids 651-670, IDAPGDVFYMPKEETRKIRK (SEQ ID NO:7).

The peptide of SEQ ID NO:6 was conjugated using MAP (multiple antigen peptide) techniques. This is done by synthesizing eight copies of the peptide onto a MAP carrier core. The peptide is attached to the core at the C-terminus. The peptide of SEQ ID NO:7 was conjugated at the N-terminus using the traditional KLH method. The derivatized peptides were suitable for direct injection for antibody production along with adjuvant

The peptide-hapten constructs were emulsified with Freund's incomplete adjuvant and injected into New Zealand white female rabbits housed in individual cages. Two rabbits were utilized for each immunogen. The antibodies were generated in the rabbits over a 10 week period, during which the antibody titer was monitored by ELISA on all bleeds and at the end of the protocol.

The antibody raised against the KLH-derivatized peptide was purified prior to further analysis. The MTA1 antibody in raw rabbit serum was dialyzed into 1×PBS with 10% BSA (bovine serum albumin) and 0.1% NaAz (sodium azide) for use and storage.

### Example 2

### Method of Constructing a Tissue Array

A tissue array for immunohistochemical analysis of MTA1 was constructed by using pre-weighed samples of powdered breast tumor tissue that were removed from frozen storage (−70 Celsius), hydrated with chilled 1× phosphate buffered saline solution, and quickly pelleted by centrifugation. The tissue pellet was fixed in buffered formalin, and embedded into paraffin blocks.

To assemble the arrays, cores were obtained from the tissue blocks with a 5 mm dermal biopsy punch. The cores were re-embedded in an alphanumeric grid pattern (12 samples per block) into a pre-cored blank paraffin block. An orientation marker denoting the A1 sample was included on each block, and consisted of either a section of normal endocervix or myometrium tissues. A microtome was used to cut 3 micron sections from the tissue array paraffin blocks, which were transferred to glass microscope slides. The resulting slides were then deparrafinized and subjected to standard IHC protocols.

### Example 3

### Method of Constructing His-Tagged MTA1

A vector encoding for MTA1 polypeptide having a histidine tag, named MTA1-T7, was constructed as described by Mazumdar et al., 2001. The vector used for the construction was the pcDNA 3.1/His A construct available from Invitrogen. The vector was modified with a T7 tag, which was inserted directly before the translation start site. The full length human MTA1 construct (SEQ ID NO:2) was then subcloned into the multiple cloning site. Detection studies were done in this case using an antibody to the T7 tag (Novagen).

### Example 4

### MTA1 Wester Blot Analysis

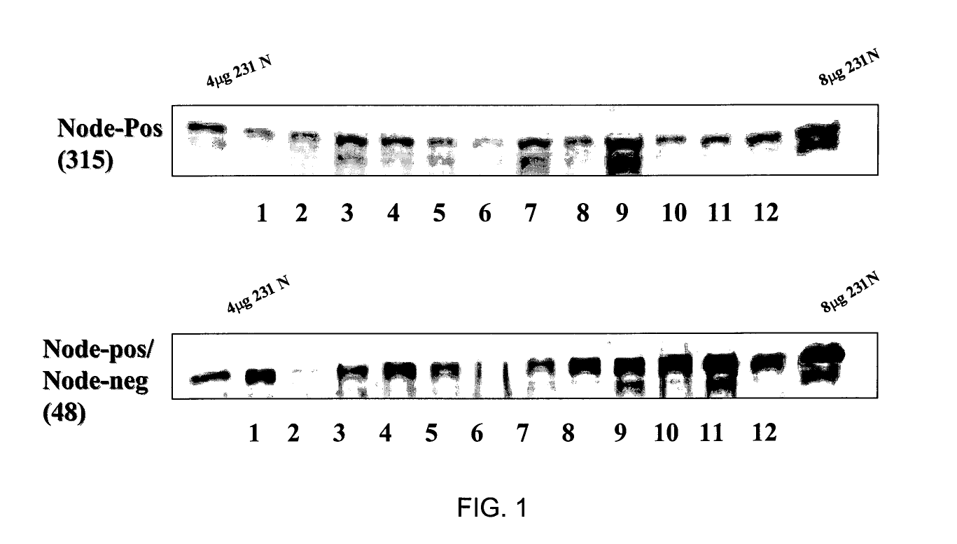

MTA1 western blot analysis was performed using a specific anti-MTA1 antibody and the results are shown in FIG. 1. The top row is analysis on node-positive breast tumor lysates, which were taken from patients who either received endocrine therapy (n=186), or did not receive endocrine therapy (n=119). The bottom row is a MTA1 western blot analysis on breast tumor lysates from node-negative patients (n=25), patients diagnosed with 1-3 positive nodes (n=17), and patients diagnosed with more than 3 positive nodes (n=7). Outcome data on these patients was not obtained. Internal controls were run on each gel in the first and last lanes in which 4 ug and 8 ug of MDA-MB-231 nuclear extract was loaded, respectively. The internal controls were used to normalize expression levels between multiple gels.

Table 2 summarizes the statistical results of the Western blot analysis. The calculated Spearman coefficients indicate that MTA1 protein levels correlate with protein levels of ER, NCOR, AIB1, SAFB in node-positive breast tumor tissues. Interestingly, MTA1 protein levels did not strongly correlate to the node-positive factor. Overall, the data indicated that substantial heterogeneity existed in the levels of MTA1 protein expression between tumors from different patients. This analysis also indicated that the selectivity, efficiency and accuracy of the specific anti-MTA1 antibody was very good and suitable for immunohistochemical analysis of MTA1 protein levels.

TABLE 2
MTA1 Western Blot Results

| Node-Positive Bank | | | Node-Positive/Node-Negative | | |
|---|---|---|---|---|---|
| (n = 315) | | | Bank (n = 48) | | |
| | Spearman | | | Spearman | |
| Factor | Coefficent | P value | Factor | Coefficent | P value |
| ER | 0.25475 | <0.0001 | ER | 0.42725 | 0.0025 |
| NCOR | 0.50312 | <0.0001 | SAFB | 0.55008 | <0.0001 |
| AIB1 | 0.37162 | <0.0001 | IRS1 | 0.29182 | 0.0442 |
| FKHR | 0.29023 | <0.0001 | IRS2 | 0.28793 | 0.0472 |
| SAFB | 0.38852 | <0.0001 | POSITVE | −0.13693 | 0.3534 |
| | | | NODE | | |
| HER2 | 0.14382 | 0.0107 | TUMOR | −0.07208 | 0.6380 |
| | | | SIZE | | |
| TUMOR SIZE | −0.08803 | 0.1195 | S PHASE | −0.02901 | 0.8448 |
| S PHASE | 0.03929 | 0.4914 | | | |

ER = estrogen receptor;
PR = progesterone receptor;
NCOR = NCOR = nuclear receptor corepressor;
AIB1 = amplified in breast cancer 1;
FKHR = Forkhead, Drosophila, homolog of, in rhabdomyosarcoma;
SAFB = Scaffold attachment factor B;
HER2 = Tyrosine kinase-type cell surface receptor her2 (also referred to in the art as ERBB2);
POSITVE NODE = Axillary lymph node positive breast cancer;
TUMOR SIZE = increasing tumor size, in centimeters;
S PHASE = fraction of tumor cells in the DNA synthesis (S) phase of the mitotic cell cycle (in percent).

### Example 5

### MTA1 Cell Fractionations

The specific anti-MTA1 antibody was further characterized by cell fractionation of different breast carcinoma cell lines. Cell fractionations for endogenous MTA1 expression in the cell lines was determined using the anti-MTA1 antibody generated using SEQ ID NO:6, and results are shown in FIGS. 2A to 2D. In each case, MTA1 fractionates completely into the nucleus of the cell lines. Actin was used as a loading control, and total histone H1 was used as a nuclear marker for the fractionations.

The second band observed in the nuclear fraction for MTA1 indicates alternative splicing occurs at the C-terminus, and because the anti-MTA1 antibody was prepared from a N-terminus peptide sequence, the antibody is detecting both polypeptides. Thus, the MTA1 polypeptide that is measured includes structural variants, which result from expression at the 14q locus.

The data obtained using the anti-MTA1 antibody correlated with the fractionation data taken from cells transiently transfected with the T7-tagged MTA1/His construct and probed with the anti-T7 antibody (Novagen). The data confirmed the MTA1 signal was nuclear, and this nuclear localization provided a basis for reading immunohistochemical (IHC) studies using tissue arrays.

In FIG. 2, T=total lysate, C=cytoplasm extract, N=nuclear extract.

### Example 6

### Tissue Array Analysis of MTA1 Expression

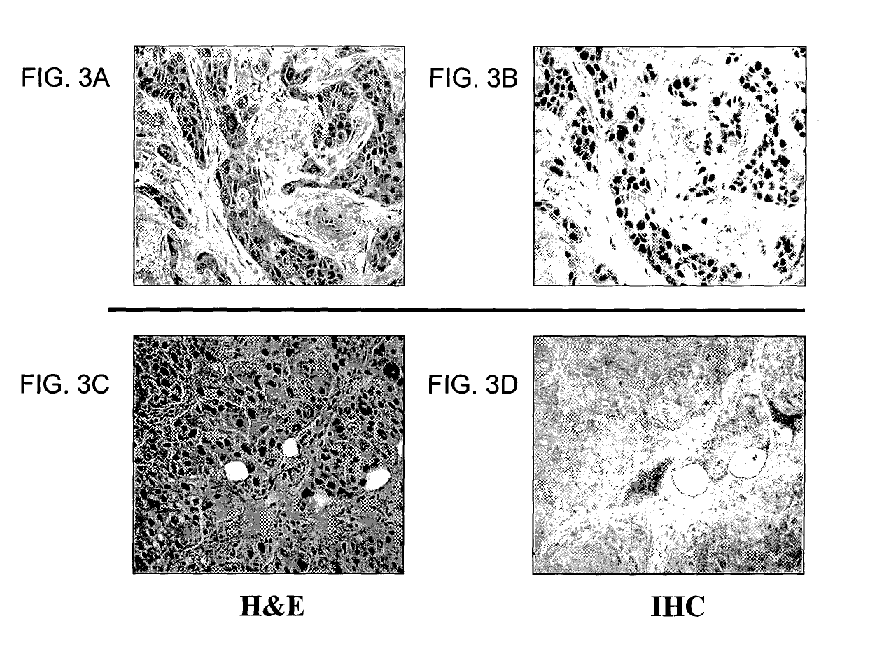

Using the prepared breast cancer tissue arrays, MTA1 staining was performed. FIGS. 3A to 3D shows the different patterns of staining that occurred in two different tumor types: invasive (FIGS. 3A-B) and normal (FIGS. 3C-D) Shown are the haematoxylin & eosin (H&E) histology (FIGS. 3A and 3C), in which the nuclei stain blue and the other components stain shades of red and pink, and the corresponding MTA1 IHC (FIGS. 3B and 3D). The tumor tissue shown in FIGS. 3A-B is representative of a positive staining for MTA1, and the tumor tissue shown in FIGS. 3C-D is representative of a negative staining for MTA1 expression.

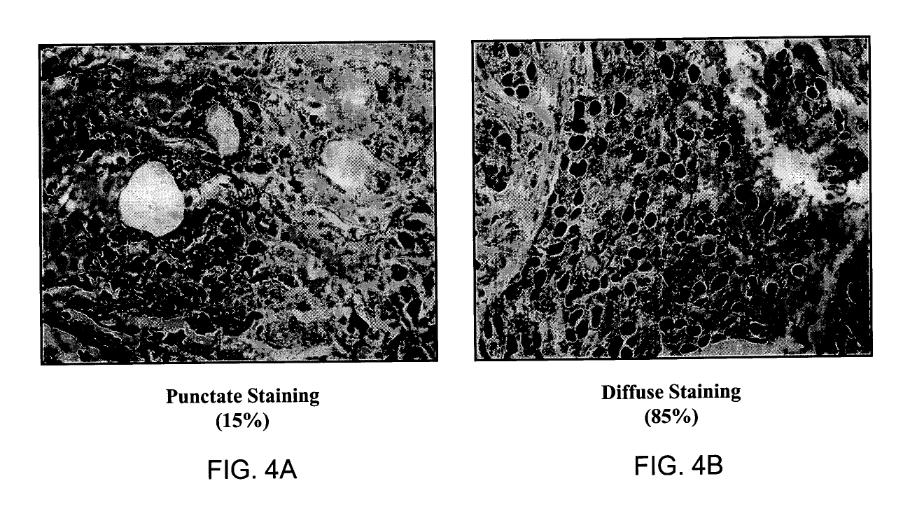

FIG. 4A illustrates the distinctive punctate staining that was observed in approximately 15% of the cases analyzed. The diffuse staining illustrated in FIG. 4B was observed in about 85% of the cases analyzed. For purposes of scoring, the differences in the patterns were not considered.

However, in certain embodiments, this pattern observation is incorporated as a third element to the IHC scoring system. Doing such involves determining, qualitatively, the quality relative to the quantity of signal obtained by nuclear staining for MTA1 polypeptide; assigning a value to the range of patterning observed defined by the punctati diffuse patterns, and statistically evaluating the total scores.

### Example 7

### IHC Scoring

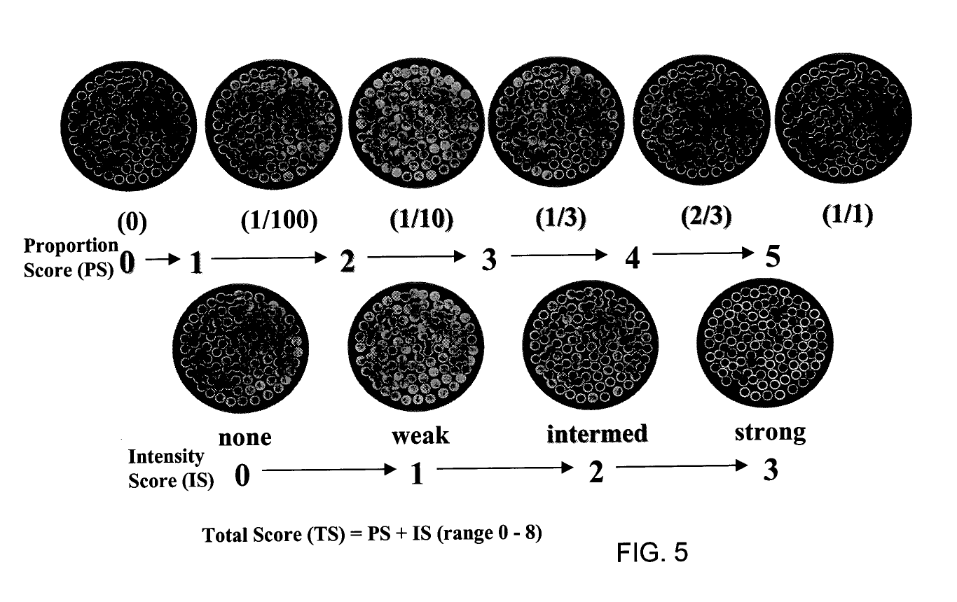

The semi-quantitative IHC scoring system that was used is described by Allred et al., 1993 and is illustrated in FIG. 5. A qualitative determination of the proportion score (PS), which is shown on the top line, was made. The proportion score involves the amount of stained cells relative to the amount of unstained cells. The intensity score (IS) was determined, which is indicative of the amount of MTA1 protein that is present at the stained site. The total score (TS) is the sum total of the proportion score and the intensity score: TS=PS+IS

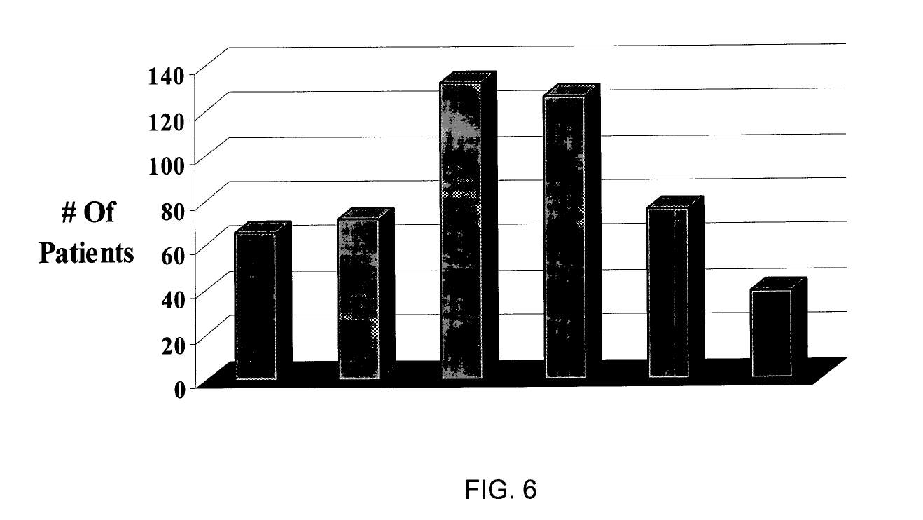

The total score of the MTA1 IHC scoring analysis was graphed and is shown in FIG. 6. The graph indicates that MTA1 expression is heterogeneous in the population tested, and the heterogeneity assumes a normal distribution. Statistical analysis for the IHC analysis indicates that MTA1 is not remeasuring the known prognostic and/or predictive factors of positive node, tumor size and S phase (Table 3).

ER=estrogen receptor; PR=progesterone receptor; POSITIVE NODE=Axillary lymph node positive breast cancer; TUMOR SIZE=Increasing tumor size, in centimeters; S PHASE=Fraction of tumor cells in the DNA synthesis (S) phase of the mitotic cell cycle (in percent).

TABLE 3
MTA1 IHC Correlations

| | Spearman | |
|---|---|---|
| Factor | Coefficient | P value |
| ER | 0.27994 | <0.0001 |
| PR | 0.11207 | 0.0001 |
| Positive node | 0.00735 | 0.8691 |
| Tumor size | −0.03995 | 0.3770 |
| S phase | 0.00481 | 0.9187 |

### Example 8

### Multivariate Analysis

Multivariate analysis was used on the data obtained from all patients (n=491), in which 162 recurrences occurred, and from the untreated patients (n=186). The results are summarized in Tables 4 and 5. MTA1 shows a strong correlation with decreased disease-free survival (e.g., increased MTA1 expression correlates with a decrease in disease-free survival). The presence of positive nodes was the only factor that correlated stronger with disease-free survival than MTA1 expression. Specifically, the hazard ratio for MTA1 as a predictive factor in untreated patients indicates that the patient has about a 30% chance of recurrence and in all patients indicates that the patient has about a 17% chance of recurrence. In fact, for untreated patients, MTA1 is a better predictor of disease-free survival than tumor size. However, MTA1 does not directly correlate with the presence of positive nodes, thereby indicating that MTA1 is a prognostic factor that is independent of nodal status. Consequently, high levels of MTA1 protein are particularly useful for determining disease-free survival in node-negative breast cancers because, in part, the MTA1 protein level functions as a marker for micrometastasis.

The PR factor is known to be slightly favorable and this observation is observed, indicating that no statistical bias exists in the statistical determinations. The multivariate analysis data shown in Table 4 comprises analysis of all patients (N=997, 326 recurrences) using cutpoint score of 5 and in Table 5 comprises analysis of all node-negative patients (N=577, 128 recurrences) using cutpoint score of 5. Chemo=chemotherapy; Endo=endocrine therapy.

TABLE 4
Multivariate Analysis of All Patients (n = 491)

| | Univariate | Multivariate | |
|---|---|---|---|
| Factor | p-value | p-value | Hazard ratio |
| Positive node | <.0001 | <.0001 | 2.249 |
| MTA1 | 0.5656 | 0.0015 | 1.544 |
| S phase | <.0001 | 0.0866 | 1.142 |
| Endo | 0.6544 | 0.0058 | 0.700 |
| Chemo | <.0001 | 0.0083 | 0.688 |
| ER | <.0001 | 0.0006 | 0.646 |
| PR | 0.0001 | 0.1917 | n/a |
| Tumor size | <.0001 | 0.0002 | 1.379 |
| Ploidy | 0.1716 | 0.2679 | n/a |

TABLE 5
Multivariate Analysis of Untreated Patients (n = 186)

| | Univariate | Multivariate | |
|---|---|---|---|
| Factor | p-value | p-value | Hazard ratio |
| MTA1 | 0.0151 | 0.0067 | 1.672 |
| S Phase | 0.0022 | 0.0052 | 1.357 |
| Endo | 0.1455 | 0.0932 | 0.695 |
| Chemo | 0.3459 | 0.7089 | n/a |
| ER | 0.1341 | 0.7271 | n/a |
| PR | 0.4300 | 0.8064 | n/a |
| Tumor size | 0.1674 | 0.3280 | n/a |
| Ploidy | 0.1319 | 0.6899 | n/a |

### Example 9

### MTA1 and Disease-Free Survival

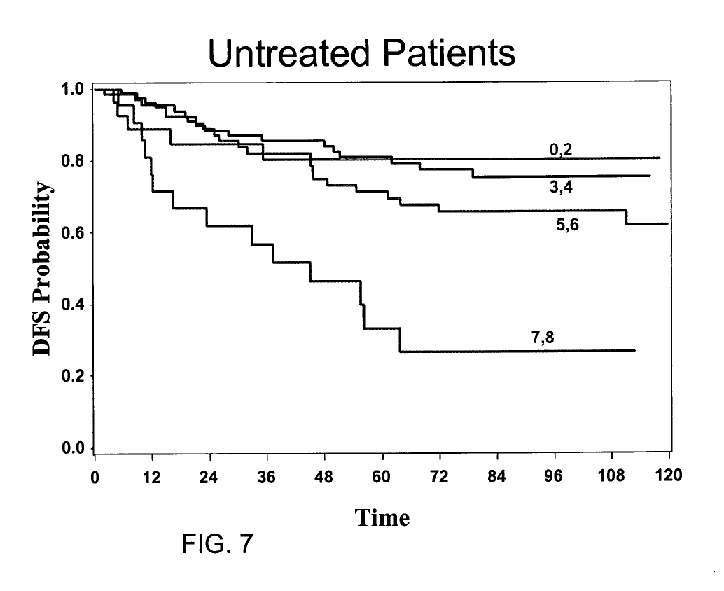

The disease-free survival probability was determined for the untreated patients data pool based on MTA1 expression (e.g., MTA1 protein levels) and given as a Kaplan-Meyer curve (FIG. 7) in which MTA1 is a continuous variable. The analysis is given for a 10 year post-diagnosis time period, and the numbers on the lines indicate the total IHC score that produces the respective disease-free survival probabilities. The analysis indicates that high levels of MTA1 protein correlate with a substantial decrease in disease-free survival over time. For example, the total IHC scores of 7 and 8 indicate that a proportion score is a minimum of about 50% of the nuclear staining and the associated intensity is strong, and these patients fitting this determination have a decreased disease-free survival.

### Example 10

### Patient Population and Tumor Specimens

The subjects in this study derive originally from a large tumor bank and associated clinical database representing primarily community-based patients who had hormone receptor assays performed in our laboratory (Tumor Bank and Data Network Core of the Breast Center of Baylor College of Medicine). To facilitate immunohistochemical study of prognostic factors, a cohort of 614 node-negative, primary tumor samples was assembled into a low density (12 cores per block) paraffin embedded tissue array. Using previously described methods, frozen, pulverized samples were first fixed and paraffin embedded, and then arrayed. Cases selected for inclusion in the tissue array were diagnosed between 1973 and 1993 with stage 1 or 2 primary breast cancer, treated with mastectomy or lumpectomy plus axillary dissection, with or without post-operative radiation therapy, no distant metastases, no neoadjuvant therapy, and with complete data on tumor size, receptors, use and type of adjuvant therapy, and S-phase fraction. ERα (ER-6F11, Novocastra, Newcastle upon Tyne, UK) and PgR (PgR.1294, Dako, Carpinteria, Calif.) were previously analyzed by immunohistochemistry using standard protocols. Cases with Allred scores for nuclear staining of 3-8 were deemed positive for ERα or PgR expression. S-phase fraction was previously measured on all tumors in the cohort using DNA flow cytometry (31). Cases were classified as low (<6% S-phase), intermediate (6-10%), or high (>10%) proliferation.

### Example 11

### Immunohistochemistry

Immunohistochemistry was performed on histological sections made from 100 μg pulverized samples of stage 1 and 2 primary breast tumors, and control tissue. Standard 3-4 μm sections were cut from the blocks, mounted onto slides, then deparaffinized and hydrated in a routine manner. Antigen retrieval was performed in a pressure cooker with 10 mM citrate buffer, pH3, for 5 minutes. The slides were removed and rinsed in six changes of 1×PBS. Endogenous peroxidase was blocked with 3% hydrogen peroxide solution (TBS, 30% hydrogen peroxide, sodium azide) for 5 minutes. Endogenous biotin was blocked with an Avidin Biotin Blocking kit for 15 minutes (Vector, Burlingame, Calif.). Slides were incubated with the MTA1 primary antibody for 1 hour at room temperature at a dilution of 1:800, then rinsed in TBS-20 (TBS-Tween-20). Slides were next incubated with swine anti-rabbit biotinylated secondary antibody diluted 1:200 (Dako, Carpinteria, Calif.) for 30 minutes at room temperature, and then rinsed in TBS-20 (TBS-Tween-20). Peroxidase-conjugated streptavidin (Dako, Carpinteria, Calif.) was applied to the slides for 30 minutes at room temperature at a dilution of 1:200. After rinsing, the slides were treated with DAB+ solution (Dako, Carpinteria, Calif.) for 15 minutes, rinsed in H₂O, then treated with 0.2% osmium tetroxide for 30 seconds for chromogen enhancement. Slides were rinsed in H₂O, counterstained with 0.05% methyl green, and routinely dehydrated in ethanol and cleared in xylene for mounting in Cytoseal (vWR, West Chester, Pa.). Immunostained slides were evaluated by light microscopy for the presence of nuclear staining, and scored.

### Example 12

### Statistical Analysis

Spearman rank correlations were used to characterize associations between MTA1 immunohistochemistry scores (0-8) and other prognostic factors. Survival analysis was used to examine the association between MTA1 and relapse-free survival or overall survival. Relapse-free survival (RFS) was defined as the time from diagnosis to first recurrence (local or distant), or to last contact or death (censored). Overall survival (OS) was defined as the time from diagnosis to death from any cause or last contact (censored). For purposes of analysis, receptors were dichotomized (Allred score 3 or more, versus 0 or 2) as positive or negative and coded as 1 or 0 respectively. Tumor size (≦2 cm, >2-5 cm, >5 cm) and S-phase (<6%, 6-10%, and >10%) were trichotomized, and coded 0, 1, and 2, respectively and treated as single continuous variables in survival analyses. These cut-offs and code schemes have been used widely. In preliminary analyses, the assumption of proportional hazards was verified for MTA1, and an analysis of functional form suggested that MTA1 scores above 5 are associated with proportionately increasing risk, while scores of 5 or less carry essentially the same good risk. Therefore, MTA1 values of 0-5, 6, 7, and 8 were coded as 0, 1, 2, and 3 respectively in the analysis and treated as a single continuous variable. Kaplan Meier estimates and log rank tests were used to display and test the univariate association between RFS or OS and MTA1. Cox proportional hazards regression was used to test the independent contribution of MTA1 after accounting for other potentially important covariates. Models were built with forward stepwise selection with the significance level set to 0.1. Adjusted survival curves were generated for various values of MTA1 using Cox regression estimates, with cohort averages being used for other covariates in the model. Plots have been truncated at 120 months for graphical presentation, but all data were included in the analyses. Analyses were performed using the SAS (Version 8.2, Cary N.C.), and Splus (Version 6.1, Insightful, Seattle, Wash.).

### Example 13

### MTA1 Expression in Immunohistochemical Studies of Archival Breast Tumors

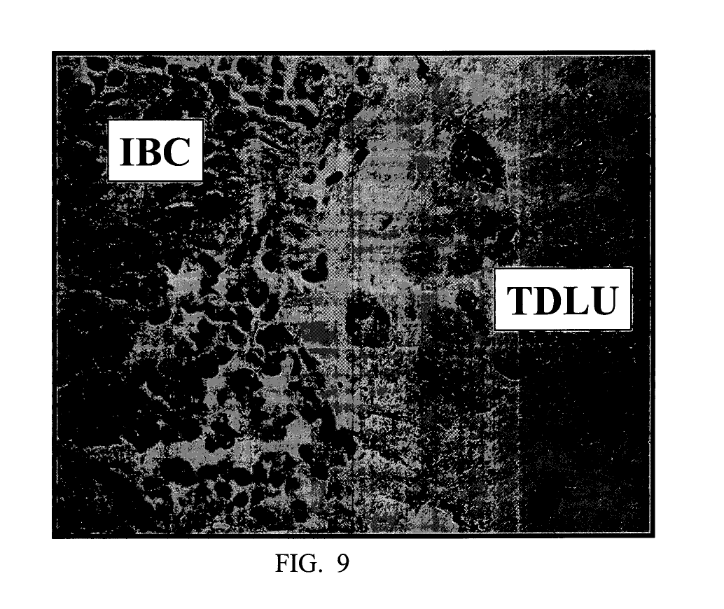

Patient and tumor characteristics for the study population are shown in Table 6. No patients had histologic evidence of metastases in the axillary lymph nodes at diagnosis. 397 of the patients received no systemic adjuvant treatment after surgery, while 217 patients received endocrine therapy, chemotherapy, or both. Median follow-up time for patients remaining disease-free was seven years. Expression of MTA1 was evaluated by immunohistochemistry and nuclear signal was quantified by the proportion of positive cells and by staining intensity (9). A typical case is shown in FIG. 9 in which high MTA1 expression is observed in the nucleus of invasive breast cancer (IBC). In contrast, an adjacent normal terminal ductal lobular unit (TDLU) is negative for MTA1 expression. Of the 614 specimens analyzed, 561 (92%) showed some positive immunostaining (IHC scores >0), with total scores ranging from 0 and 2-8. The median IHC score was 5.

TABLE 6
Patient and Tumor Characteristics

| | |
|---|---|
| Untreated | Treated |

| Variable | N | % | N | % |
|---|---|---|---|---|
| All Patients | 397 | 100% | 217 | 100% |
| Age at Diagnosis | | | | |
| ≧50 yr | 95 | 24% | 63 | 29% |
| <50 yr | 302 | 76% | 154 | 71% |
| Tumor Size | | | | |
| ≦2 cm | 173 | 43% | 98 | 45% |
| >2 to 5 cm | 205 | 52% | 112 | 52% |
| >5 cm | 19 | 5% | 7 | 3% |
| S-Phase Fraction | | | | |
| Low (<6%) | 134 | 34% | 68 | 31% |
| Intermediate (6-10%) | 97 | 24% | 63 | 29% |
| High (>10%) | 166 | 42% | 86 | 40% |
| ER□ | | | | |
| Positive (Total Score ≧3) | 260 | 65% | 155 | 71% |
| Negative (Total Score <3) | 137 | 35% | 62 | 29% |
| PgR | | | | |
| Positive (Total Score ≧3) | 195 | 49% | 132 | 61% |
| Negative (Total Score <3) | 202 | 51% | 85 | 39% |
| Adjuvant Therapy | | | | |
| None | 397 | 100% | | |
| Endocrine Only | | | 154 | 71% |
| Chemotherapy Only | | | 41 | 19% |
| Both | | | 22 | 13% |
| Relapse Status | | | | |
| Disease-Free | 295 | 74% | 184 | 85% |
| Relapsed | 102 | 26% | 33 | 15% |
| Vital Status | | | | |
| Alive | 257 | 65% | 183 | 84% |
| Dead | 140 | 35% | 34 | 16% |
| MTA1 Total IHC Score | | | | |
| 0 | 40 | 10% | 13 | 6% |
| 2 | 4 | 1% | 1 | 0% |
| 3 | 67 | 17% | 26 | 12% |
| 4 | 104 | 26% | 46 | 21% |
| 5 | 91 | 23% | 60 | 28% |
| 6 | 62 | 16% | 46 | 21% |
| 7 | 26 | 7% | 22 | 10% |
| 8 | 3 | 1% | 3 | 1% |

### Example 14

### Relationship of MTA1 Expression to Other Prognostic Factors

Five other prognostic factors had been previously analyzed in these specimens independent of this study, and the data were correlated with MTA1 protein expression (Table 7). MTA1 levels were significantly positively correlated with those of ERα and PgR in both treated and untreated cases, although the correlations were relatively weak. MTA1 protein levels did not correlate with tumor size or s-phase fraction, known markers of early disease recurrence, and correlations with age were weak and variable in direction.

TABLE 7
Spearman rank correlations of MTA1 with other breast
cancer-related factors.

| | |
|---|---|
| Untreated | Treated |
| (N = 397) | (N = 217) |

| Variable | rₛₚ | P value | rₛₚ | P value |
|---|---|---|---|---|
| ER□ | 0.26 | <0.001 | 0.33 | <0.001 |
| PgR | 0.15 | 0.003 | 0.30 | <0.001 |
| Age | −0.10 | 0.05 | 0.27 | <0.001 |
| Tumor Size | 0.03 | 0.52 | −0.12 | 0.07 |
| S phase fraction | 0.08 | 0.09 | −0.03 | 0.64 |

### Example 15

### Relationship of MTA1 to Clinical Outcome in Node-Negative Breast Cancer

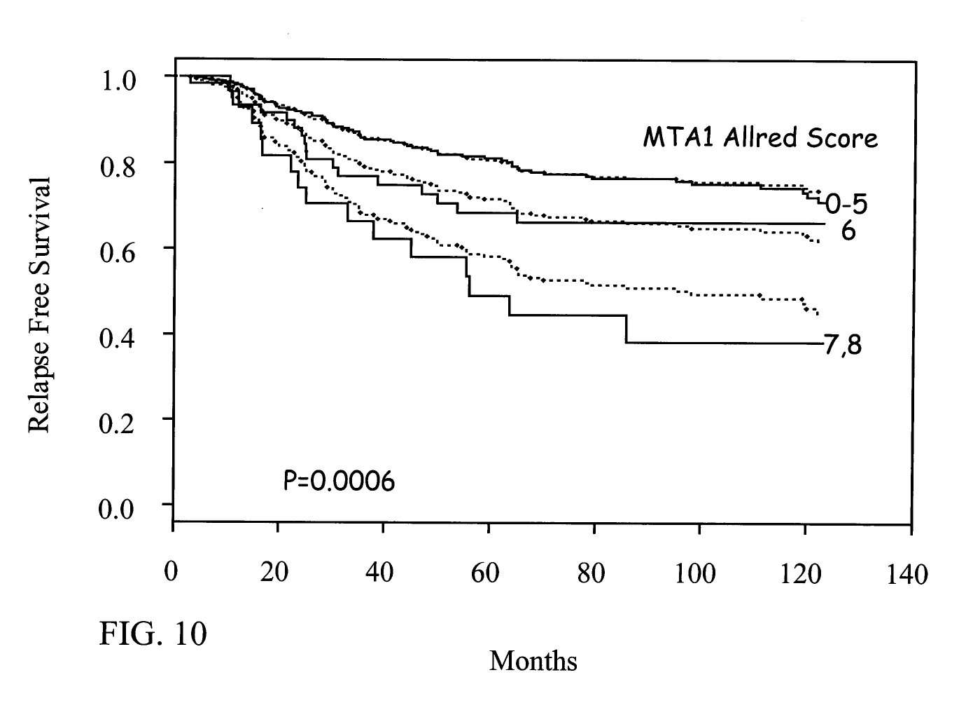

Univariate survival analyses and multivariable Cox regression were used to examine the association between MTA1 protein levels and relapse-free or overall survival. In both univariate and multivariable analysis of relapse-free survival in untreated patients, high expression of MTA1 was associated with an increased risk of early disease recurrence (P≦0.0017) (FIG. 10). Indeed, MTA1 was the strongest predictor of early disease recurrence, even greater than tumor size and s-phase fraction; ERα and PgR were not predictive. These results did not extend to overall survival where tumor size was the only significant variable. In the treated subset of patients, MTA1 was no longer significant (Table 8), suggesting that treatment effects may be associated with MTA1 expression.

TABLE 8
Survival Analysis of MTA1 for Relapse-free and Overall Survival.

| | Relapse Free Survival | | | Overall Survival | | |
|---|---|---|---|---|---|---|
| | | Cox Regression | | | Cox Regression | |
| | Univariate | Multivariable | HazardRatio³ | Univariate | Multivariable | HazardRatio³ |
| Variable | P Value¹ | P Value² | (95% CI) | P Value¹ | P Value² | (95% CI) |
| Untreated | | | | | | |
| MTA1 | 0.0002 | 0.0006 | 1.55 (1.21-2.00) | 0.46 | 0.41 | |
| S-phase | 0.007 | 0.071 | 1.25 (0.98-1.60) | 0.36 | 0.51 | |
| Tumor size | 0.01 | 0.02 | 1.41 (1.02-1.95) | 0.04 | 0.09 | 1.27 (0.96-1.67) |
| ER□ | 0.55 | 0.59 | | 0.34 | 0.51 | |
| PgR | 0.93 | 0.30 | | 0.57 | 0.67 | |
| Treated | | | | | | |
| MTA1 | 0.75 | 0.61 | | 0.36 | | |
| S-phase | 0.03 | 0.04 | 1.58 (1.02-2.42) | 0.20 | | |
| Tumor size | 0.89 | >0.99 | | 0.34 | | |
| ER□ | 0.10 | 0.37 | | 0.54 | | |
| PgR | 0.10 | 0.26 | | 0.11 | | |

¹P value from the log rank test.
²P value from the Wald test of Cox regression coefficient. Stepwise selection criteria set at P = 0.1.
³Hazard Ratios are shown for a one level increase in the associated covariate, relative to baseline.

### Example 16

### MTA1 and Cancer Detection

Determining MTA1 protein levels is also contemplated as a prognostic marker for the presence of primary cancers that do not, yet, involve metastasis. The data indicates that MTA1 protein levels is a strong predictor of recurrence, and, thus, it is reasonable to expect that MTA1 protein levels are strong predictors of the initial cancer occurrence. To analyze the risk of breast cancer recurrence, immunohistochemical analysis is carried out using the specific anti-MTA1 antibodies described herein. The test is performed on formalin-fixed, paraffin-embedded primary cancer tissues obtained either during routine diagnosis (e.g., fine needle or fluid aspirates, core needle biopsies) or during surgical treatment of primary cancer (e.g., excisional biopsy or surgery). The tissue samples are subjected to immunohistochemical analysis using the specific anti-MTA1 antibodies. The IHC scoring system, illustrated in FIG. 5, is used to semi-quantitatively measure MTA1 protein levels in the cancer tissue.

Analysis of MTA1 expression and breast cancer recurrence in 577 axillary node negative breast cancers (clinical follow-up median 8.7 years) is summarized in Table 5. Using a cut point of MTA1 IHC intensity score equal to or greater than five (MTA1 nuc_ts5, on a scale of 0-8), univariate analysis showed that the node-negative breast tumors exhibited a significantly shorter disease-free survival (P=0.0151). In multivariate analysis, high MTA1 was associated with a 1.672-fold increased risk of recurrence (P=0.0067). This analysis indicates that IHC testing of primary breast cancer tissue with the MTA1-specific antibody identifies a subset of patients at high-risk for breast cancer recurrence in this otherwise low-risk group of patients.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### REFERENCES

The following references, to the extent that they provide exemplary procedural or other details supplementary to those set forth herein, are specifically incorporated herein by reference.

Patents

U.S. Pat. No. 3,817,837
U.S. Pat. No. 3,850,752
U.S. Pat. No. 3,939,350;
U.S. Pat. No. 3,996,345
U.S. Pat. No. 4,277,437
U.S. Pat. No. 4,275,149
U.S. Pat. No. 4,366,241
U.S. Pat. No. 4,472,509
U.S. Pat. No. 4,938,948
U.S. Pat. No. 4,816,567
U.S. Pat. No. 4,376,110
U.S. Pat. No. 5,821,337
U.S. Pat. No. 5,670,488
U.S. Pat. No. 4,676,980
U.S. Pat. No. 5,705,629
U.S. Pat. No. 4,683,202
U.S. Pat. No. 4,682,195,
U.S. Pat. No. 5,645,897
U.S. Pat. No. 5,399,346
WO 91/00360
WO 92/200373
EP 03089
EP 266,032
WO 93/06213
WO 84/03564
   Publications
Allred D C, Clark G M, Elledge R, Fuqua S A, Brown R W, Chamness G C, Osborne C K, and McGuire W L. Association of p53 protein expression with tumor cell proliferation rate and clinical outcome in node-negative breast cancer. J Natl Cancer Inst 1993 Feb. 3; 85(3): 176-7.
Dear, T. N., McDonald, D. A., Kefford, R. F., Transcriptional down-regulation of a rat gene, WDNM2, in metastatic DMBA-8 cells, 1989, Cancer Res. 49:5323-5328.
Mahoney M G, Simpson A, Jost M, Noe M, Kari C, Pepe D, Choi Y W, Uitto J, Rodeck U., Metastasis-associated protein (MTA) 1 enhances migration, invasion, and anchorage-independent survival of immortalized human keratinocytes, 2002, Oncogene 28:21(14):2161-2170.
Martin M D, Hopp T A, Clark G M, Osterreich S, Allred DC, Osborne C K, and O'Connell, P. Characterization and Expression of a Chromosome 14q Metastasis Gene in Breast Cancer. Proceedings of the American Association for Cancer Research 43: 735, 2002.
Mazumdar A, Wang R A, Mishra S K, Adam L, Bagheri-Yarmand R, Mandal M, Vadlarnudi R K, Kumar R., Transcriptional repression of oestrogen receptor by metastasis-associated protein 1 corepressor, 2001, Nat Cell Biol 3(1):30-7.
Dressler L G, Eudey L, Gray R, Torrney D C, McGuire W L, Gilchrist K W, Clark G M, Osborne C K, Mansour E G, Abeloff M D (1992). Prognostic potential of DNA flow cytometry measurements in node-negative breast cancer patients: preliminary analysis of an intergroup study (INT 0076), J Natl Cancer Inst Monogrl 1: 167-72
Brown R W, Allred C D, Clark G M, Osborne C K, Hilsenbeck S G (1996). Prognostic value of Ki-67 compared to S-phase fraction in axillary node-negative breast cancer. Clin Cancer Res 2:585-92.
Schirrmacher, V., Adv. Cancer Res. 43:1-73, 1985; Liotta, et al., Cell 64(2):327-336, 1991.
Allegra et al., 1979, Cancer Treat. Rep. 63: 1271-1277
Von Rosen et al., 1989, Breast Cancer Res. Treat. 11: 23-32
Fischer et al., 1992, J. Natl. Cancer Inst. 11: 152-258
Froehler et al., 1986.
Clark et al., 1994, N. Engl. J. Med. 320: 627-633
Fischer et al., 1993, in Cancer Medicine, 3d ed., Holland et al., eds., Philadelphia: Lea & Febiger, pp. 1706-1774
Early Risk Cancer Trialists, Corraborative Group, Lancet, 1992, 339 (8784): 1-15
Ludwig Breast Cancer Study Group, 1989, N. Engl. J. Med. 320: 491-496.
Gasparini et al., 1993, J. Natl. Cancer Inst. 85: 1206-1219
Tam, J P (1988), Proc Natl Acad Sci USA 85; 5409-5413.
Posnett, D N et al (1988) Journal Biol Sci 263; 1719-1725.
Tam J P, Zavala F (1989) Jour Immunol Meth 124; 53-61.
Tam J P, Lu Y A (1989) Proc Natl Acad Sci USA 86; 9084-9088.
Lad, K et al (1989) J. Dental Research, Abstract 68; 405.
Sanatarpia RP et al (1988) Archives Oral Biol 33;567.
Chang, CC et al (1990) European Peptide Symp Proc.
Goding, Monoclonal Antibodies: Principles and Practice, pp. 59-103 (Academic Press, 1986) Kozbor, J. Immunol. (1984), and Brodeur, et al., Monoclonal Antibody Production Techniques and Applications, pp. 51-63 (Marcel Dekker, Inc., New York, 1987
Johnson, Kevin S. and Chiswell, David J., Current Opinion in Structural Biology 3, 564-571 (1993)
Clackson et al., Nature 352, 624-628 (1991)
Doolittle M H and Ben-Zeev O, 1999
Gulbis B and Galand P, 1993
De Jager R et al., 1993
Nakamura et al., 1987
Nagase T, Ishikawa K, Kikuno R, Hirosawa M, Nomura N, Ohara O (1999) Prediction of the coding sequences of unidentified human genes. XV. The complete sequences of 100 new cDNA clones from brain which code for large proteins in vitro. DNA Res 29:337-45
Rivera E, Holmes F A, Buzdar A U, Asmar L, Kau S W, Fraschini G, Walters R, Theriault R L, Hortobagyi G N (2002). Fluorouracil, doxorubicin, and cyclophosphamide followed by tamoxifen as adjuvant treatment for patients with stage IV breast cancer with no evidence of disease. Breast J 8: 2-9
Howell A, Howell S J, Clarke R, Anderson E (2001). Where do selective estrogen receptor modulators (SERMs) and aromatase inhibitors (AIs) now fit into breast cancer treatment algorithms? J Steroid Biochem Mol Biol 79:227-37
Sambrook, Fritsch, Maniatis, In: Molecular Cloning: A Laboratory Manual, Vol. 1, Cold Spring Harbor Laboratory Press, Cold Spring Harbor, N.Y., Ch. 7, 7.19-17.29, 1989.
Remington's Pharmaceutical Sciences, 18th Ed. Mack Printing Company, 1990

1. A method of providing a prognosis of disease-free survival in a cancer patient comprising:
obtaining a sample from said patient;
measuring MTA1 polypeptide level in said sample; and
predicting said disease-free survival based upon the measured level of said MTA1 polypeptide, wherein when said level of MTA1 polypeptide is increased as compared to a control sample, said disease-free survival is decreased.

2. The method of claim 1, wherein the patient has epithelial-derived cancer.

3. The method of claim 2, wherein the patient has a cancer selected from the group of breast cancer, esophagus cancer, colon cancer, gastric cancer, skin cancer, liver cancer, pancreatic cancer, bladder cancer, ovarian cancer, cervical cancer, testicular cancer, brain cancer and lung cancer.

4. The method of claim 3, wherein the cancer is breast cancer.

5. The method of claim 1, wherein the sample comprises a fluid, a tissue or a cell.

6. The method of claim 1, wherein the MTA1 polypeptide is SEQ ID NO:1 or a structural variant thereof.

7. The method of claim 1, wherein the level is measured using an anti-MTA1 antibody that binds to a specific MTA1 peptide sequence

8. The method of claim 7, wherein the anti-MTA1 antibody selectively cross-reacts with SEQ ID NO: 1 or a structural variant thereof.

9. The method of claim 8, wherein the anti-MTA1 antibody binds to SEQ ID NO:6.

10. A method of prognosticating metastasis in a cancer patient comprising:
obtaining a sample from said patient;
measuring MTA1 polypeptide level in the said sample;
determining a prognosis based upon the measured level of said MTA1 polypeptide, wherein an increased level of MTA1 polypeptide as compared to a control sample indicates a poor prognosis.

11. A method of prognosticating micrometastasis in a cancer patient comprising:
obtaining a sample of said patient;
measuring MTA1 polypeptide level in said sample;
determining a prognosis based upon the measured level of said MTA1 polypeptide, wherein an increased level of MTA1 polypeptide as compared to a normal level indicates a poor prognosis.

12. A composition of matter comprising an anti-MTA1 antibody that selectively cross-reacts with a MTA1 polypeptide.

13. The composition of claim 12, wherein the MTA1 polypeptide is SEQ ID NO:1 or a structural variant thereof.

14. The composition of claim 12, wherein the anti-MTA1 antibody binds to a specific MTA1 peptide sequence.

15. The composition of claim 14, wherein the specific MTA1 peptide sequence is SEQ ID NO:6.

16. The composition of claim 12, wherein the anti-MTA1 antibody is a polyclonal antibody or a monoclonal antibody.

17. A kit comprising an anti-MTA1 antibody in a container, wherein said antibody selectively cross-reacts with an MTA1 polypeptide.

18. The kit of claim 17, wherein the MTA1 polypeptide is SEQ ID NO:1 or a structural variant thereof.

19. The kit of claim 17, wherein the anti-MTA1 antibody binds to a specific MTA1 peptide sequence.

20. The kit of claim 17, wherein the anti-MTA1 antibody binds to SEQ ID NO:6.

21. A method of treating a cancer patient comprising administering to said patient a therapeutically effective amount of a therapeutic agent that decreases MTA1 polypeptide level.

22. The method of claim 21, wherein the MTA1 polypeptide comprises SEQ ID NO:1 or a structural variant thereof.

23. The method of claim 21, wherein the therapeutic agent interferes with translation of the MTA1 polypeptide.

24. The method of claim 21, wherein the therapeutic agent comprises an oligonucleotide complementary to a polynucleotide sequence encoding a specific MTA1 peptide sequence.

25. The method of claim 21, wherein the therapeutic agent neutralizes biological activity of the MTA1 polypeptide.

26. The method of claim 21, wherein the therapeutic agent comprises a specific MTA1 peptide sequence.

27. A method of screening for a candidate therapeutic agent that improves disease-free survival in a cancer patient comprising:
introducing to a cell a test agent, wherein said cell has a polynucleotide encoding a MTA1 polypeptide or a structural variant thereof which is under control of a promoter that is operable in said cell; and
measuring a level of the MTA1 polypeptide or the structural variant thereof after introduction of the test agent, wherein when said measured level is decreased following the introduction, the test agent is the candidate therapeutic agent.

28. The method of claim 27, wherein the cell is a cancer cell.

29. The method of claim 27, wherein the therapeutic agent is an antagonist of MTA1.

30. The method of claim 27, wherein the therapeutic agent interferes with translation of the MTA1 polypeptide.

31. The method of claim 30, wherein the therapeutic agent is an antisense oligonucleotide.

32. The method of claim 31, wherein said antisense oligonucleotide is complementary to a polynucleotide sequence encoding a specific MTA1 peptide sequence.

33. The method of claim 27, wherein the therapeutic agent binds to the MTA1 polypeptide or structural variant, wherein said binding neutralizes the biological activity of the MTA1 polypeptide or structural variant thereof.
